(19) United States
(12) Patent Application Publication
(10) Pub. No.: US 2024/0052062 A1
(43) Pub. Date: Feb. 15, 2024

(54) REDUCING SURGERY-ASSOCIATED HEMOLYSIS IN COLD AGGLUTININ DISEASE PATIENTS

(71) Applicant: Bioverativ USA Inc., Waltham, MA (US)

(72) Inventors: William E. Hobbs, Waltham, MA (US); Michael J. Storek, Cambridge, MA (US); Tor Henrik Anderson Tvedt, Hønefoss (NO); Marek Wardecki, Kielpin (PL); Nancy Wong, North Andover, MA (US)

(73) Assignee: Bioverativ USA Inc., Waltham, MA (US)

(21) Appl. No.: 18/477,981

(22) Filed: Sep. 29, 2023

Related U.S. Application Data
(63) Continuation of application No. PCT/US2022/022745, filed on Mar. 31, 2022.
(60) Provisional application No. 63/168,986, filed on Mar. 31, 2021.

(51) Int. Cl. C07K 16/40; A61P 7/00

(52) U.S. Cl. CPC C07K 16/40; A61P 7/00; C12Y 304/21042

(57) ABSTRACT

Provided herein are methods for reducing or preventing surgery-associated hemolysis in subjects who have cold agglutinin disease (CAD) (e.g., been diagnosed with CAD or have had at least one symptom of CAD).

### RELATED APPLICATION

This application claims priority to U.S. provisional application Ser. No. 63/168,986, filed Mar. 31, 2021, the entire content of which is incorporated herein by reference.

### FIELD

The present application relates to methods for reducing surgery-associated hemolysis.

### BACKGROUND

Cold agglutinin disease (CAD) is a rare, chronic type of autoimmune hemolytic anemia, where hemolysis is driven by classical complement pathway activation. Complement activation assures the rapid initiation of the complement cascade as a part of an early immune response. CAD is primarily a disease of the elderly, with a mean age at diagnosis of 67 years, and even higher median ages are observed in studies exploring therapy for CAD. A recent retrospective study observed that a high proportion of CAD patients require treatment, and that approximately 40% experience hemolytic exacerbations. Due to the chronic nature of CAD and age of these patients, the risk of comorbidities requiring medical treatment is significant.

### SUMMARY

Major surgery poses a critical threat to patients with CAD due to an increase in hemolysis and a significant drop in hemoglobin levels, mediated through complement activation, For example, patients undergoing cardiac surgery using extracorporeal circulation are prone to complement-induced hemolysis and/or cold agglutinin related clogging affecting the heart and other critical organs. Not only are patients with clinically overt CAD at risk, but also those with CAD manifested by mild signs and symptoms. In addition to having greater risk of complications during surgeries, CAD patients may also be at risk of being disqualified from elective procedures in the absence of safe and effective measures to prevent surgery-associated hemolysis.

Data from the study described herein demonstrates that cardiac surgery, even accompanied by extracorporeal circulation, can be performed on a CAD patient without exacerbating hemolysis, when the surgery is timed such that the patient's serum levels of sutimlimab are predicted to be higher than (e.g., four times higher than) what is understood in the field to be needed to sufficiently suppress hemolysis, accounting for the dilution of circulating sutimlimab expected to occur during the surgery.

Thus, some aspects of the present disclosure provide a method of reducing or preventing hemolysis in a subject in need thereof undergoing a major surgery, comprising maintaining in the subject a therapeutic serum concentration of a proximal classical complement pathway inhibitor (e.g., a C1s inhibitor, such as sutimlimab), wherein the subject has CAD (e.g., has been diagnosed with CAD or has had at least one symptom of CAD) and the therapeutic serum concentration is effective to reduce or prevent hemolysis.

In some embodiments, the maintaining comprises administering to the subject a maintenance dose of the proximal classical complement pathway inhibitor before the subject undergoes the major surgery, wherein the dose is effective to maintain the therapeutic serum concentration during the major surgery.

In some embodiments, the maintaining comprises administering to the subject the maintenance dose of the proximal classical complement pathway inhibitor within seven days of the subject undergoing the major surgery. In some embodiments, the maintaining comprises administering to the subject the maintenance dose of the proximal classical complement pathway inhibitor within three days, within two days, or within one day of the subject undergoing the major surgery,

In some embodiments, the method further comprises assessing in the subject a therapeutic serum concentration of the proximal classical complement pathway inhibitor.

In some embodiments, the maintaining comprises administering to the subject at least one additional dose of the proximal classical complement pathway inhibitor before, during and/or after the subject undergoes the major surgery.

Other aspects of the present disclosure provide a method of reducing or preventing hemolysis in a subject in need thereof undergoing a major surgery, comprising performing the major surgery on the subject when the subject has a proximal classical complement pathway inhibitor serum concentration effective to reduce or prevent hemolysis, wherein the subject has CAD (e.g., been diagnosed with cold agglutinin disease CAD or has had at least one symptom of CAD) and has been undergoing treatment with the proximal classical complement pathway inhibitor.

In some embodiments, the treatment comprises administration of at least one loading dose and at least one maintenance dose of the proximal classical complement pathway inhibitor.

In some embodiments, the method comprises (a) administering to the subject at least one maintenance dose of the proximal classical complement pathway inhibitor, and (b) performing the major surgery on the subject within seven days of administering the at least one maintenance dose of the proximal classical complement pathway inhibitor.

In some embodiments, the method further comprises, prior to (a), administering to the subject at least one loading dose of a proximal classical complement pathway inhibitor.

In some embodiments, (b) comprises performing the major surgery on the subject within three days, within two days, or within one day of administering the at least one maintenance dose of the proximal classical complement pathway inhibitor.

Yet other aspects of the present disclosure provide a method of reducing or preventing hemolysis in a subject in need thereof undergoing a major surgery, comprising assessing a serum concentration of a proximal classical complement pathway inhibitor in a subject who has CAD (e.g., has been diagnosed with CAD) and is undergoing treatment with a proximal classical complement pathway inhibitor, and performing the major surgery on the subject within seven days of the assessing.

In some embodiments, the method comprises performing the major surgery on the subject within three days, within two days, or within one day of the assessing,

In some embodiments, the method comprises assessing the serum concentration of the proximal classical complement pathway inhibitor before, during, and/or after the major surgery.

In some embodiments, the method further comprises administering to the subject at least one dose of the proximal classical complement pathway inhibitor before, during and/or after performing the major surgery on the subject.

In some embodiments, the subject has been diagnosed with CAD.

In some embodiments, the major surgery is a major cardiac surgery. For example, the major cardiac surgery may be coronary artery bypass graft (CABG) surgery.

In some embodiments, the major surgery is associated with a drop in body temperature and/or hypoxia.

In some embodiments, the major surgery involves hemodilution and/or extracorporeal circulation.

In some embodiments, the proximal classical complement pathway inhibitor is a C1s inhibitor, a C1r inhibitor, a C1q inhibitor, a C2 inhibitor, or a C4 inhibitor. In some embodiments, the proximal classical complement pathway inhibitor is a C1s inhibitor,

In some embodiments, the inhibitor is an antibody.

In some embodiments, the anti-C1s antibody comprises a heavy chain (FIC) complementarity determining region 1 (CDR1) comprising the amino acid sequence of SEQ ID NO: 5, an HC complementarity determining region 2 (CDR2) comprising the amino acid sequence of SEQ ID NO: 6, an FIC, complementarity determining region 3 (CDR3) comprising the amino acid sequence of SEQ ID NO: 7, a light chain (LC) CDR1 that comprises the amino acid sequence of SEQ ID NO: 8, an LC CDR2 comprising the amino acid sequence of SEQ ID NO: 9, and an LC CDR3 comprising the amino acid sequence of SEQ ID NO: 10.

In some embodiments, the anti-C1s antibody comprises a heavy chain variable region (VH) comprising the amino acid sequence of SEQ ID NO: 3 and comprises a light chain variable region (VL) comprising the amino acid sequence of SEQ ID NO: 4.

In some embodiments, the antibody is sutimlimab. Sutimlimab comprises an HC comprising the amino acid sequence of SEQ ID NO: 1 and an LC comprising the amino acid sequence of SEQ ID NO: 2.

In some embodiments, the anti-C1s antibody comprises an HC CDR1 comprising the amino acid sequence of SEQ ID NO: 15, an HC CDR2 comprising the amino acid sequence of SEQ ID NO: 16, an HC CDR3 comprising the amino acid sequence of SEQ ID NO: 17, an LC CDR1 comprising the amino acid sequence of SEQ ID NO: 18, an LC CDR2 comprising the amino acid sequence of SEQ ID NO: 19, and an LC CDR3 comprising the amino acid sequence of SEQ ID NO: 20.

In some embodiments, the anti--C1s antibody comprises a VH comprising the amino acid sequence of SEQ ID NO: 13 and comprises a VL comprising the amino acid sequence of SEQ ID NO: 14.

In some embodiments, the anti-C1s antibody comprises an HC comprising the amino acid sequence of SEQ ID NO: 11 and an LC comprising the amino acid sequence of SEQ ID NO: 12.

In some embodiments, the anti-C1s antibody comprises an IgG4 constant region.

In some embodiments, sutimlimab is administered in an amount of about 5 grams to about 8 grams. In some embodiments, sutimlimab is administered in an amount of about 6.5 grams to about 7.5 grams. In some embodiments, the subject weighs less than 75 kilograms, and the amount is 6.5 grams, or the subject weighs 75 kilograms or more, and the amount is 7.5 grams.

In some embodiments, the therapeutic serum concentration of the inhibitor is at least 100 μg/mL.

Each of International Publication Nos. WO 2014/071206, filed Nov. 2, 2012, entitled Anti-Complement C1s Antibodies and Uses Thereof, WO 2016/164358, filed Apr. 6, 2015, entitled Humanized Anti-C1s Antibodies and Methods of Use Thereof and in WO 2018/170145, filed Mar. 14, 2017, entitled Methods for Treating Complement-Mediated Diseases and Disorders is herein incorporated by reference in its entirety.

### BRIEF DESCRIPTION OF THE DRAWINGS

FIGS. 1A-1D show PK simulation data for sutimlimab in a CAD patient enrolled in the Phase 3 Cardinal trial, an open-label, single-arm study of sutimlimab, FIG. 1A demonstrates the predicted and observed peak and trough levels of sutimlimab during the study period. FIG. 1B shows the predicted sutimlimab level without surgery , while FIGS. 1C and 1D show the predicted drop in sutimlimab level if the patient underwent coronary artery bypass grafting (CABG) with standard extracorporeal circulation (ECC) in 2 days or 7 days after sutimlimab infusion, respectively.

FIG. 2 is a timeline showing the most important blood samples and actions, prior to, during, and after the operation and the extracorporeal circulation (ECC).

### DETAILED DESCRIPTION

The complement system is a well-known effector mechanism of the immune response, providing not only protection against pathogens and other harmful agents but also recovery from injury. The classical complement pathway is triggered by activation of the first component of complement, referred to as the C1 complex, which includes C1q, C1r, and C1s proteins. Upon binding of C1 to an immune complex, the C1s component, a diisopropyl fluorophosphate (DFP)-sensitive serine protease, cleaves complement components C4 and C2 to initiate activation of the classical complement pathway. The classical complement pathway plays a role in cold agglutinin disease, for example.

Cold agglutinins (CAs) are autoantibodies, usually of the IgM class, directed against surface proteins expressed on red blood cells (RBCs). Most CAs are of low clinical significance since their thermal amplitudes (e.g., the optimal temperature of reactivity with RBC antigens) are at temperatures that are not physiologically relevant. However, CAs with thermal amplitudes greater than 28-30° C. are often associated with clinically relevant effects. CAs are capable of causing complications through two independent mechanisms. Firstly, agglutination of RBCs in acral parts of the body can result in reduced microcirculation, vasospasm, and acrocyanosis. Secondly, once bound to an antigen on the RBC surface, the CA-antigen complex is an avid activator of the classical complement pathway via binding the C1 complex, the consequence of which is hemolytic anemia primarily from extra vascular RBC destruction and modest intravascular hemolysis through terminal complement pathway activation. Primary cold agglutinin disease (CAD) is characterized by chronic hemolysis, the presence of CAs, and evidence of complement-mediated RBC destruction (C3d present on RBCs).

The low level of complement proteins available under steady state conditions limits the rate of RBC destruction. Hence, many patients with CAD are only mildly anemic, but approximately half of all patients with CAD require transfusions at some time point. However, after an infection, trauma, or surgery, the 100- to 1000-fold increase in complement activity seen during the acute phase response exacerbates hemolysis, leading to a significant drop in hemoglobin levels. Coronary heart surgery poses particular challenges to patients with CAD, both through hypothermia during extracorporeal cardiopulmonary bypass resulting in increased CA binding to RBC antigens which causes severe agglutination in the heart or other vital organs, and through an increase in hemolysis caused by the increase in complement activation.

### Methods of Reducing or Preventing Hemolysis

The present disclosure is based, in part, on evidence demonstrating that major surgery-associated hemolysis in subjects with CAD can be reduced or even prevented both intra-operatively as well as post-operatively by pre-operatively administering a proximal classical complement pathway inhibitor (e.g., a C1s inhibitor). By maintaining a therapeutic concentration of a proximal classical complement pathway inhibitor (e.g., a C1s inhibitor) before, during, and/or after surgery, surgery-associated hemolysis can be safely and efficiently reduced or prevented in a subject who has CAD (e.g., has been diagnosed with CAD or has had at least one symptom of CAD). Surgery-associated hemolysis includes both hemolysis induced directly by the act of the surgery itself as well as hemolysis induced indirectly by external conditions associated with the surgery including, but not limited to, extracorporeal circulation, hemodilution, temperature of fluids administered intravenously and temperature of the operating room.

Accordingly, in one aspect, the present disclosure provides a method of reducing or preventing hemolysis in a subject in need thereof undergoing a major surgery, comprising maintaining in the subject a therapeutic serum concentration of a proximal classical complement pathway inhibitor, wherein the subject has CAD (e.g., has been diagnosed with CAD or has had at least one symptom of CAD) and the therapeutic serum concentration is effective to reduce or prevent hemolysis.

In some embodiments, the method comprises maintaining in the subject a therapeutic serum concentration of the proximal classical complement pathway inhibitor before, during and/or after the major surgery. The therapeutic serum concentrations will depend, at least in part, on the C1s inhibitor. “Maintenance” of a therapeutic serum concentration of the C1s inhibitor is achieved when the serum concentration is at a level known to inhibit complement pathway activity (e.g., at least 50% inhibition, at least 60% inhibition, at least 70% inhibition, at least 80% inhibition, or at least 90% inhibition of complement pathway activity), For example, for sutimlimab, maintenance of a therapeutic concentration is achieved when the serum concentration of sutimlimab does not fall below 20 μg/mL. Sutimlimab concentration at 20 μg/ml has been associated with 90% reduction of complement pathway activity. Thus, in some embodiments, maintenance of a sutimlimab concentration includes maintenance at a concentration of 20 lag/ml to 200 μg/ml, 30 to 200 μg/ml, 40 μg/ml to 200 μg/ml, 50 μg/ml to 200 μg/ml, 60 μg/ml. to 200 μg/ml, 70 μg/ml to 200 μg/ml, 80 μg/ml. to 200 μg/ml, 90 μg/ml to 200 100 μg/ml to 200 μg/ml., 110 to 200 μg/ml, 120 to 200 μg/ml, 120 μg/ml to 200 μg/ml, 140 μg/ml to 200 μg/ml, or 150 μg/ml to 200 μg/ml. In some embodiments, maintenance of a sutimlimab concentration includes maintenance at a concentration of 20 μg/ml, 30 μg/ml, 40 μg/ml, 50 μg/ml, 60 μg/ml, 70 μg/ml, 80 μg/ml, 90 μg/ml, 100 μg/ml, 110 μg/ml, 120 μg/ml, 130 μg/ml, 140 μg/ml, 150 μg/ml, 160 μg/ml, 170 μg/ml, 180 μg/ml, 190 μg/ml, or 200 μg/ml.

In some embodiments, maintaining a therapeutic serum concentration comprises administering to the subject a maintenance dose of the proximal classical complement pathway inhibitor before the subject undergoes the major surgery, wherein the dose is effective to maintain the therapeutic serum concentration during the major surgery. A maintenance dose is a dose of the proximal classical complement pathway inhibitor administered at a regular dosing interval to maintain the therapeutic serum concentration of the proximal classical complement pathway inhibitor.

In some embodiments, a maintenance dose of the proximal classical complement pathway inhibitor (e.g., a C1s inhibitor) is administered to the subject directly before the subject undergoes surgery, within 1-24 hours before the subject undergoes surgery, or within 2-14 days before the subject undergoes surgery. In some embodiments, the maintenance dose of the inhibitor is administered directly before surgery. In some embodiments, the maintenance dose of the inhibitor is administered within 1, 2, 3, 4, 5, 6, 7, 8, 9, 10, 11, 12, 13, 14, 15, 16, 17, 18, 19, 20, 21, 22, 23, or 24 hours before surgery. In some embodiments, the maintenance dose of the inhibitor is administered within 2, 3, 4, 5, 6, 7, 8, 9, 10,11, 12, 13, or 14 days before surgery. In some embodiments, the maintenance dose of the inhibitor is administered 1 day before surgery. In some embodiments, the maintenance dose of the inhibitor is administered 2 days before surgery. In some embodiments, the maintenance dose of the inhibitor is administered 3 days before surgery. In some embodiments, the maintenance dose of the inhibitor is administered 7 days before surgery. In some embodiments, the maintenance dose of the inhibitor is administered 1-2, 1-3, 1-4, 1-5-6, 1-7, 2-3, 2-4, 2-5, 2-6, 2-7, 3-4, 3-5, 3-6, 3-7, 3-10, 4-7, 5-7, 5-10, 5-14, 6-14, 7-14, 8-14, 9-14, or 10-14 days before surgery. In some embodiments, the maintenance dose of the inhibitor is administered 1-2 days before surgery. In some embodiments, the maintenance dose of the inhibitor is administered 1-3 days before surgery. In some embodiments, the maintenance dose of the inhibitor is administered 1-7 days before surgery.

In some embodiments, the maintaining comprises administering to the subject at least one additional dose (e.g., 1, 2, or 3 doses) of the proximal classical complement pathway inhibitor (e.g., a C1s inhibitor) before, during and/or after the subject undergoes the major surgery,

In another aspect, the present disclosure provides a method of reducing or preventing hemolysis in a subject in need thereof undergoing a major surgery, comprising performing the major surgery on the subject when the subject has a proximal classical complement pathway inhibitor (e.g., a C1s inhibitor) serum concentration effective to reduce or prevent.

hemolysis, wherein the subject has CAD (e.g., has been diagnosed with CAD or has had at least one symptom of CAD) and has been undergoing treatment with the proximal classical complement pathway inhibitor. In some embodiments, the major surgery is performed when the subject's serum levels of the inhibitor are predicted to be two times, three times, or four times higher than what is needed to sufficiently reduce or prevent hemolysis. In some embodiments, the major surgery is performed when the subject's serum levels of the inhibitor are four times higher than what is needed to sufficiently reduce or prevent hemolysis.

In yet another aspect, the present disclosure provides a method of reducing or preventing hemolysis in a subject in need thereof undergoing a major surgery, comprising: assessing a serum concentration of a proximal classical complement pathway inhibitor (e.g., a C1s inhibitor) in a subject who has CAD (e.g., has been diagnosed with CAD) and is undergoing treatment with a proximal classical complement pathway inhibitor; and performing the major surgery after assessing the serum concentration of the proximal classical complement pathway inhibitor.

In some embodiments, treatment with the proximal classical complement pathway inhibitor comprises administration of at least one loading dose and at least one maintenance dose of the proximal classical complement pathway inhibitor (e.g., a C1s inhibitor). In some embodiments, the inhibitor is administered as one or more loading doses followed by one or more maintenance doses at dosing intervals,

A loading dose is an initial dose that is administered at the beginning of a course of treatment with a proximal classical complement pathway inhibitor. When more than one loading dose is administered, the loading doses can be administered 7 days apart, 14 days apart, 21 days apart, 28 days apart, two months apart, three months apart, or four months apart. In some embodiments, the loading doses are administered 7 days apart. In some embodiments, the loading dose is a different dosage amount than the maintenance administered at dosing intervals. In some embodiments, the loading dose is the same dosage amount as the maintenance dose administered at dosing intervals.

When more than one maintenance dose is administered, the maintenance doses can be administered at a dosing interval of five days, six days, seven days, eight days, nine days, ten days, eleven days, twelve days, thirteen days, fourteen days, fifteen days, sixteen days, seventeen days, eighteen days, nineteen days, twenty days, twenty one days, twenty two days, twenty three days, twenty four days, twenty five days, twenty six days, twenty seven days, twenty eight days, twenty nine days, thirty days, or thirty one days. In some embodiments, the maintenance doses are administered at a dosing interval of fourteen days.

In some embodiments, the treatment with the proximal classical complement pathway inhibitor comprises administration of one loading dose followed by one or more maintenance doses. In some embodiments, the treatment with the inhibitor comprises administration of two loading doses followed by one or more maintenance doses. In some embodiments, the treatment with the inhibitor comprises administering two loading doses weekly for two weeks followed by one or more maintenance doses every two weeks.

In some embodiments, the method comprises administering to the subject at least one maintenance dose (e.g., 1, 2, 3, 4, 5, or more) of the proximal classical complement pathway inhibitor (e.g., a C1s inhibitor) and performing the major surgery after administering the at least one maintenance dose of the proximal classical complement pathway inhibitor. In some embodiments, at least one loading dose e.g., 1, 2, 3, 4, 5, or more) of a proximal classical complement pathway inhibitor is administered prior to administering the at least one. maintenance dose.

In some embodiments, at least one additional dose of the proximal classical complement pathway inhibitor (e.g., a C1s inhibitor) before, during and/or after the subject undergoes the major surgery.

In some embodiments, at least one additional dose of the inhibitor is administered to the subject before the surgery. In some embodiments, an additional dose of the inhibitor is administered to the subject directly before the subject undergoes surgery, within 1-24 hours before the subject undergoes surgery, or within 2-14 days before the subject undergoes surgery. In some embodiments, an additional dose of the inhibitor of the inhibitor is administered directly before surgery. In some embodiments, an additional dose of the inhibitor is administered within 1, 3, 4, 5, 6, 7, 8, 9, 10, 11, 12, 13, 14, 15, 16, 17, 18, 19, 20, 21, 22, 23, or 24 hours before surgery. In some embodiments, an additional dose of the inhibitor is administered within 2, 3, 4, 5, 6, 7, 8, 9, 10,11, 12, 13, or 14 days before surgery. In some embodiments, an additional dose of the inhibitor is administered 1 day before surgery. In some embodiments, an additional dose of the inhibitor is administered 2 days before surgery. In some embodiments, an additional dose of the inhibitor is administered 3 days before surgery. In some embodiments, an additional dose of the inhibitor is administered 7 days before surgery. In some embodiments, an additional dose of the inhibitor is administered 1-2, 1-3, 1-4, 1-5, 1-6, 1-7, 2-3, 2-4, 2-5, 2-6, 2-7, 3-4, 3-5, 3-6, 3-7, 3-10, 4-7, 5-7, 5-10, 5-14, 6-14, 7-14, 8-14, 9-14, or 10-14 days before surgery. In some embodiments, an additional dose of the inhibitor administered 1-2 days before surgery. In some embodiments, an additional dose of the inhibitor is administered 1-3 days before surgery. In some embodiments, an additional dose of the inhibitor is administered 1-7 days before surgery.

In some embodiments, at least one additional dose of the inhibitor is administered to the subject during the major surgery. In some embodiments, an additional dose of the inhibitor is administered at the beginning of surgery. In some embodiments, an additional dose of the inhibitor is administered within an hour of commencing the surgery. In some embodiments, an additional dose of the inhibitor is administered within 2 hours of commencing the surgery. In some embodiments, the inhibitor is administered two or more times during the surgery.

In some embodiments, at least one additional dose of the inhibitor is administered to the subject after the surgery. In some embodiments, an additional dose of the inhibitor is administered to the subject directly after surgery, 1-24 hours after surgery, or 1-14 days after surgery. In some embodiments, an additional dose of the inhibitor is administered directly after surgery. In some embodiments, an additional dose of the inhibitor is administered 1, 2, 3, 4, 5, 6, 7, 8, 9, 10, 11, 12, 13, 14, 15, 16, 17, 18, 19, 20, 21, 22, 23, or 24 hours after surgery. In some embodiments, an additional dose of the inhibitor is administered 1, 2, 3, 4, 5, 6, 7, 8, 9, 10, 11, 12, 13, or 14 days after surgery. In some embodiments, an additional dose of the inhibitor is administered 1 day after surgery. In some embodiments, an additional dose of the inhibitor is administered 2 days after surgery. In some embodiments, an additional dose of the inhibitor is administered 7 days after surgery. In some embodiments, an additional dose of the inhibitor is administered 1-2, 1-3, 1-4, 1-5, 1-6, 1-7, 2-3, 2-4, 2-5, 2-6, 2-7, 3-4, 3-5, 3-6, 3-7, 3-10, 4-7, 5-7, 5-10, 5-14, 6-14, 7-14, 8-14, 9-14, or 10-14 days after surgery. In some embodiments, an additional dose of the inhibitor is administered 1-2 days after surgery. In some embodiments, an additional dose of the inhibitor is administered 1-7 days after surgery. In some embodiments, the inhibitor is administered two or more times after the surgery.

In some embodiments, the surgery is performed directly after the maintenance dose is administered, within 1-24 hours after the maintenance dose is administered, or within 2-14 days after the maintenance dose is administered. In some embodiments, the surgery is performed directly after the maintenance dose is administered. In some embodiments, the surgery is performed within 1, 2, 3, 4, 5, 6, 7, 8, 9, 10, 11, 12, 13, 14, 15, 16, 17, 18, 19, 20, 21, 22, 23, or 24 hours after the maintenance dose is administered. In some embodiments, the surgery is performed within 2, 3, 4, 5, 6, 7, 8, 9, 10, 11, 12, 13, or 14 days after the maintenance dose is administered. In some embodiments, the surgery is performed 1 day after the maintenance dose is administered. In some embodiments, the surgery is performed 2 days after the maintenance dose is administered. In some embodiments, the surgery is performed 3 days after the maintenance dose is administered. In some embodiments, the surgery is performed 7 days after the maintenance dose is administered. In some embodiments, the surgery is performed 1-2, 1-3, 1-4, 1-5, 1-6, 1-7, 2-3, 2-4, 2-5, 2-6, 2-7, 3-4, 3-5, 3-6, 3-7, 3-10, 4-7, 5-7, 5-10, 5-14, 6-14, 7-14, 8-14, 9-14, or 10-14 days after the maintenance dose is administered. In some embodiments, the surgery is performed 1-2 days after the maintenance dose is administered. In some embodiments, the surgery is performed. 1-3 days after the maintenance dose is administered. In some embodiments, the surgery is performed 1-7 days after the maintenance dose is administered.

In some embodiments, the methods of the disclosure comprise assessing the serum concentration of the proximal classical complement pathway inhibitor (e.g., a C1s inhibitor) in the subject. The serum concentration of the inhibitor may be assessed before, during, and/or after the major surgery.

In some embodiments, the serum concentration of the inhibitor is assessed before the surgery. In some embodiments, the serum concentration of the inhibitor is assessed directly before the subject undergoes surgery, within 1-24 hours before the subject undergoes surgery, or within 2-14 days before the subject undergoes surgery. In some embodiments, the serum concentration of the inhibitor is assessed directly before surgery. In some embodiments, the serum concentration of the inhibitor is assessed within 1, 2, 3, 4, 5, 6, 7, 8, 9, 10, 11, 12, 13, 14, 15, 16, 17, 18, 19, 20, 21, 22, 23, or 24 hours before surgery. In some embodiments, the serum concentration of the inhibitor is assessed within 2, 3, 4, 5, 6, 7, 8, 9, 10, 11, 12, 13, or 14 days before surgery. In some embodiments, the serum concentration of the inhibitor is assessed 1 day before surgery. In some embodiments, the serum concentration of the inhibitor is assessed 2 days before surgery. In some embodiments, the serum concentration of the inhibitor is assessed 3 days before surgery. In some embodiments, the serum concentration of the inhibitor is assessed 7 days before surgery. In some embodiments, the serum concentration of the inhibitor is assessed 1-2, 1-3, 1-4, 1-5, 1-6, 1-7, 2-3, 2-4, 2-5, 2-6, 2-7, 3-4, 3-5, 3-6, 3-7, 3-10, 4-7, 5-7, 5-10, 5-14, 6-14, 7-14, 8-14, 9-14, or 10-14 days before surgery. In some embodiments, the serum concentration of the inhibitor is assessed 1-2 days before surgery. In some embodiments, the serum concentration of the inhibitor is assessed 1-3 days before surgery. In some embodiments, the serum concentration of the inhibitor is assessed 1-7 days before surgery.

In some embodiments, the serum concentration of the inhibitor is assessed during the major surgery. In some embodiments, the serum concentration of the inhibitor is assessed at. the beginning of surgery. In some embodiments, the serum concentration of the inhibitor is assessed within an hour of commencing the surgery. In some embodiments, the serum concentration of the inhibitor is assessed within 2 hours of commencing the surgery,

In some embodiments, the serum concentration of the inhibitor is assessed after the surgery. In some embodiments, the serum concentration of the inhibitor is assessed directly after surgery, 1-24 hours after surgery, or 1-14 days after surgery. In some embodiments, the serum concentration of the inhibitor is assessed directly after surgery. In some embodiments, the serum concentration of the inhibitor is assessed 1, 2, 3, 4, 5, 6, 7, 8, 9, 10, 11, 12, 13, 14, 15, 16, 17, 18, 19, 20, 21, 22, 23, or 24 hours after surgery. In some embodiments, the serum concentration of the inhibitor is assessed 1, 2, 3, 4, 5, 6, 7, 8. 9, 10, 11, 12, 13, or 14 days after surgery. In some embodiments, the serum concentration of the inhibitor is assessed 1 day after surgery. In some embodiments, the serum concentration of the inhibitor is assessed 2 days after surgery. In some embodiments, the serum concentration of the inhibitor is assessed 7 days after surgery. In some embodiments, the serum concentration of the inhibitor is assessed 1-2, 1-3, 1-4, 1-5, 1-6, 1-7, 2-3, 2-4, 2-5, 2-6, 2-7, 3-4, 3-5, 3-6, 3-7, 3-.10, 4-7, 5-7, 5-10, 5-14, 6-14, 7-14, 8-14, 9-14, or 10-14 days after surgery. In some embodiments, the serum concentration of the inhibitor is assessed 1-2 days after surgery. In some embodiments, the serum concentration of the inhibitor is assessed 1-7 days after surgery.

In some embodiments, the surgery is performed after assessing the serum concentration of the inhibitor. In some embodiments, the surgery is performed directly after assessing the serum concentration of the inhibitor, within 1-24 hours after assessing the serum concentration of the inhibitor, or within 2-14 days after assessing the serum concentration of the inhibitor. In some embodiments, the surgery is performed directly after assessing the serum concentration of the inhibitor. In some embodiments, the surgery is performed within 1, 2, 3, 4, 5, 6, 7, 8, 9, 10, 11, 12, 13, 14, 15, 16, 17, 18, 19, 20, 21, 22, 23, or 24 hours after assessing the serum concentration of the inhibitor. In some embodiments, the surgery is performed within 2, 3, 4, 5, 6, 7, 8, 9, 10, 11, 12, 13, or 14 days after assessing the serum concentration of the inhibitor. In some embodiments, the surgery is performed 1 day after assessing the serum concentration of the inhibitor. In some embodiments, the surgery is performed 2 days after assessing the serum concentration of the inhibitor. In some embodiments, the surgery is performed 3 days after assessing the serum concentration of the inhibitor. In some embodiments, the surgery is performed 7 days after assessing the serum concentration of the inhibitor. In some embodiments, the surgery is performed 1-2, 1-3, 1-4, 1-5, 1-6, 1-7, 2-3, 2-4, 2-5, 2-6, 2-7, 3-4, 3-5, 3-6, 3-7, 3-10, 4-7, 5-7, 5-10, 5-14, 6-14, 7-14, 8-14, 9-14, or 10-14 days after assessing the serum concentration of the inhibitor. In some embodiments, the surgery is performed 1-2 days after assessing the serum concentration of the inhibitor. In some embodiments, the surgery is performed 1-3 days after assessing the serum concentration of the inhibitor. In some embodiments, the surgery is performed 1-7 days after assessing the serum concentration of the inhibitor.

The serum concentration of the inhibitor in the subject can be measured using techniques known in the art. In some embodiments, the inhibitor is measured using a direct binding Enzyme-Linked Immunosorbent Assay (ELISA). In some embodiments, the inhibitor is measured using an indirect ELISA. In some embodiments, the inhibitor is measured using a sandwich ELISA. In some embodiments, the inhibitor is measured using a competitive ELISA. In some embodiments, assessing the serum concentration of the inhibitor comprises predicting the serum concentration. In some embodiments, predicting the serum concentration comprises performing a PK simulation.

In some embodiments, the subject has been diagnosed with CAD. CAD includes both primary and secondary CAD. In some embodiments, the diagnosis is based on one or more of: chronic hemolysis, polyspecific direct antiglobulin test (DAT) positive, monospecific DAT strongly positive for C3d, cold agglutinin titer of ≥64 at 4° C., and immunoglobulin CY (IgG) DAT≤1±. In some embodiments, a CAD diagnosis is based on ICD-10 (international Classification of Diseases Tenth Revision) coding (e.g., 2021 ICD-10-CM Diagnosis Code D59.12). In some embodiments, the subject has had at least one symptom of CAD. The symptoms of CAD include, but are not limited to, chronic hemolysis, anemia and related symptoms (e.g., dyspnea), hemoglobinuria, jaundice, circulatory symptoms, Reynaud's phenomenon, and fatigue (see, e.g., NIH National Center for Advancing Translation Sciences Genetic and Rare Diseases information Center).

Hemolysis refers to the complement-mediated lysis of red blood cells (e.g., lysis of cells due to C3b deposition). Hemolysis as used herein also includes hemolytic exacerbation or an acute increase in hemolysis from a baseline level of hemolysis. Reducing hemolysis refers to a reduction in the magnitude of hemolysis in the subject, relative to a control. In some embodiments, reducing hemolysis refers to a suppression of hemolysis by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, relative to a control. In some embodiments, reducing hemolysis refers to a suppression of hemolysis by 10% to 15%, 15% to 20%, 20% to 25%, 25% to 30%, 30% to 35%, 35% to 40%, 40% to 45%, 50% to 55%, 55% to 60%, 60% to 65%, 65% to 70%, 70% to 75%, 75% to 80%, 80% to 85%, 85% to 90%, or 90% to 95%, relative to a control. A control herein may be the level of hemolysis of a matched CAD patient (or a group of matched CAD patients) undergoing a major surgery who has not been administered an effective amount of a proximal classical complement pathway inhibitor (e.g., an anti-C1s antibody). CAD patients are considered matched if they share certain characteristics such as sex, age, weight, height, race, severity of CAD, or any combination of the foregoing.

In some embodiments, the reduced levels of hemolysis are within 5%, 10%, 15%, 20%, 25%, 30%, 40%, 45%, 50%, 55%, 60%, 65%, 70%, 75%, 80%, 85%, 90%, or 95% of a control. In some embodiments, the reduced levels of hemolysis are within 10% to 15%, 15% to 20%, 20% to 25%, 25% to 30%, 30% to 35%, 35% to 40%, 40% to 45%, 50% to 55%, 55% to 60%, 60% to 65%, 65% to 70%, 70% to 75%, or 75% to 80% of a control. A control in this context may be the level of hemolysis in a matched healthy subject (or a group of matched healthy subjects). Healthy subjects are considered matched if they share certain characteristics such as sex, age, weight, height, race, or any combination of the foregoing.

A change in the degree of hemolysis may be measured, for example, by separating plasma from red blood cells and analyzing the amount of cell-free hemoglobin using a spectrometer (Han, V. et al. Vox Sang. 98, 116-123 (2010)). As another example, the degree of hemolysis may he actively measured by combining nanofilters, which actively filter the plasma from the red blood cells, and optofluidic sensors for evanescent absorption detection (Zhou, C. et al. ACS Sens. 3, 784-791 (2018)). Alternatively, a change in the degree of hemolysis may be monitored in real time during the major surgery by measuring the electrical resistance of the blood (see, e.g., Van Buren T. et al, Scientific Reports 10: 5101 (2020)). The degree of hemolysis may be measured based on the color of serum collected intraoperatively.

In some embodiments, the degree of hemolysis is measured by assessing the levels of one or more markers that indicate the extent of hemolysis. In some embodiments, the degree of hemolysis is measured by assessing the levels of one or more of bilirubin, lactate dehydrogenase (LDH), hemoglobin, and haptoglobin. Other methods of measuring a change in hemolysis are contemplated herein.

In some embodiments, the methods of the disclosure reduce or prevent surgery-associated hemolytic anemia. Hemolytic anemia refers to anemia due to the destruction of red blood cells. Reducing hemolytic anemia refers to a reduction in the magnitude of hemolytic anemia in the subject, relative to a control. In some embodiments, reducing hemolytic anemia refers to a suppression of hemolytic anemia by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, relative to a control. In some embodiments, reducing hemolytic anemia refers to a suppression of hemolytic anemia by 10% to 15%, 15% to 20%, 20% to 25%, 25% to 30%, 30% to 35%, 35% to 40%, 40% to 45%, 50% to 55%, 55% to 60%, 60% to 65%, 65% to 70%, 70% to 75%, 75% to 80%, 80% to 85%, 85% to 90%, or 90% to 95%, relative to a control. A control herein may be the level of hemolysis of a matched CAD patient (or a group of matched CAD patients) undergoing a major surgery who has not been administered an effective amount of a proximal classical complement pathway inhibitor (e.g., are anti-C1s antibody). CAD patients are considered matched if they share certain characteristics such as sex, age, weight, height, race, severity of CAD, or any combination of the foregoing.

In some embodiments, the reduced levels of hemolytic anemia are within 5%, 10%, 15%, 20%, 25%, 30%, 40%, 45%, 50%, 55%, 60%, 65%, 70%, 75%, 80%, 85%, 90%, or 95% of a control. In some embodiments, the reduced levels of hemolytic anemia are within 10% to 15%, 15% to 20%, 20% to 25%, 25% to 30%, 30% to 35%, 35% to 40%, 40% to 45%, 50% to 55%, 55% to 60%, 60% to 65%, 65% to 70%, 70% to 75%, or 75% to 80% of a control. A control in this context may be the level of hemolysis in a matched healthy subject (or a group of matched healthy subjects). Healthy subjects are considered matched if they share certain characteristics such as sex, age, weight, height, race, or any combination of the foregoing.

As a result of the treatment, the inhibitor can reduce (e.g., by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of a control) or prevent surgery-associated hemolysis, hemolytic anemia, and/or reduction in serum hemoglobin level. In some embodiments, the inhibitor can reduce or prevent surgery-associated hemolysis, hemolytic anemia, and/or reduction in serum hemoglobin level for at least 1, 2, 3, 4, or 5 week(s) post-surgery. In some embodiments, the level of hemoglobin, level of haptoglobin, level of lactate dehydrogenase, level of bilirubin, and/or total complement activity (CH50) in the subject are stabilized as a result of the treatment. In some embodiments, the level of hemoglobin, level of lactate dehydrogenase, level of bilirubin, and/or total complement activity (CH50) in the subject are stabilized for at least 1, 2, 3, 4, or 5 week(s) post-surgery.

In some embodiments, the methods of the disclosure maintain serum hemoglobin levels to within 5%, 10%, 15%, 20%, 25%, 30%, 40%, 45%, 50%, 55%, 60%, 65%, 70%, 75%, 80%, 85%, 90%, or 95% of the normal range of hemoglobin levels (13.5 to 17.5 g/dL for men; 1.2.0 to 15.5 g/dL for women). In some embodiments, the serum hemoglobin levels are within 10% to 15%, 15% to 20%, 20% to 25%, 25% to 30%, 30% to 35%, 35% to 40%, 40% to 45%, 50% to 55%, 55% to 60%, 60% to 65%, 65% to 70%, 70% to 75%, or 75% to 80% of the normal range of hemoglobin levels. In some embodiments, the methods of the disclosure maintain serum hemoglobin levels at least 10.0 g/dL, at least 10.1 g/dL, at least 10.2 g/dL, at least 10.3 g/dL, at least 10.4 g/dL, at least 10.5 g/dL, at least 10.6 g/dL , at least 10.7 g/dL, at least 10.8 g/dL, at least 10.9 g/dL, at least 11.0 g/dL, at least 11.1 g/dL, at least 11.2 g/dL, at least 11.3 g/dL, at least 11.4 g/dL, at least 11.5 g/dL, at least 11.6 g/dL, at least 11.7 g/dL, at least 11.8 g/dL, at least 11.9 g/dL, at least 12.0 g/d , at least 12..1 g/dL, at least 12.2 g/dL, at least 12.3 g/dL, at least 2.4 g/dL, at least 12.5 g/dL, at least 12.6 g/dL, at least 12.7 OIL, at least 12.8 g/dL, at least 2.9 g/dL, at least 13.0 g/dL, at least 13.1 g/dL, at least 13.2 g/dL, at least 13.3 g/dL, at least 13.4 g/dL, at least 13.5 g/dL, at least 13.6 g/dL, at least 13.7 g/dL, at least 13.8 g/dL, at least 13.9 g/dL, at least 14.0 g/dL, at least 14.1 g/dL, at least 14.2 g/dL, at least 14.3 g/dL, at least 14.4 g/dL, at least 14.5 g/dL, at least 14.6 g/dL, at least 14.7 g/dL, at least 14.8 g/dL, at least 14.9 g/dL, at least 15.0 g/dL, at least 15.1 g/dL, at least 15.2 g/dL, at least 15.3 g/dL, at least 15.4 g/dL, at least 15.5 g/dL, at least 15.6 g/dL, at least 15.7 g/dL, at least 15.8 g/dL, at least 15.9 g/dL, at least 16.0 g/dL, at least 16.1 g/dL, at least 16.2 g/dL, at least 16.3 g/dL, at least 16,4 g/dL, at least 16.5 g/dL, at least 16.6 g/dL, at least 16.7 g/dL, at least 16.8 g/dL, at least 16.9 g/dL, at least 17.0 g/dL, at least 17.1 g/dL, at least 17.2 g/dL, at least 17.3 g/dL, at least 17.4 g/dL, at least 17.5 g/dL, at least 17.6 g/dL, at least 17.7 g/dL, at least 17.8 g/dL, at least 17.9 g/dL, or at least 18.0 g/dL.

In some embodiments, the methods of the disclosure maintain bilirubin levels to within 5%, 10%. 15%, 20%, 25%, 30%, 40%, 45%, 50%, 55%, 60%, 65%, 70%, 75%, 80%, 85%, 90%, or 95% of the normal range of bilirubin levels (1.2 mg/dL total bilirubin or 0.3 mg/dL direct bilirubin). In some embodiments, the bilirubin levels are within 10% to 15%, 15% to 20%, 20% to 25%, 25% to 30%, 30% to 35%, 35% to 40%, 40% to 45%, 50% to 55%, 55% to 60%, 60% to 65%, 65% to 70%, 70% to 75%, or 75% to 80% of the normal range of bilirubin levels.

In some embodiments, the methods of the disclosure maintain LDH levels to within 5%, 10%, 15%, 20%, 25%, 30%, 40%, 45%, 50%, 55%, 60%, 65%, 70%, 75%, 80%, 85%, 90%, or 95% of the normal range of LDH levels (140 units per liter (U/L) to 280 U/L). In some embodiments, the LDH levels are within 10% to 15%, 15% to 20%, 20% to 25%-, 25% to 30%, 30% to 35%, 35% to 40%, 40% to 45%, 50% to 55%, 55% to 60%, 60% to 65%, 65% to 70%, 70% to 75%, or 75% to 80% of the normal range of LDH levels.

In some embodiments, the methods of the disclosure maintain haptoglobin levels to within 5%, 10%, 15%, 20%, 25%, 30%, 40%, 45%, 50%, 55%, 60%, 65%, 70%, 75%, 80%, 85%, 90%, or 95% of the normal range of haptoglobin levels (41 to 165 mg/dL). In some embodiments, the haptoglobin levels are within 10% to 15%, 15% to 20%, 20% to 25%, 25% to 30%, 30% to 35%, 35% to 40%, 40% to 45%, 50% to 55%, 55% to 60%, 60% to 65%, 65% to 70%, 70% to 75%, or 75% to 80% of the normal range of haptoglobin levels.

In some embodiments, the methods of the disclosure maintain Cl-150 levels within 5%, 10%, 15%, 20%, 25%, 30%, 40%, 45%, 50%, 55%, 60%, 65%, 70%, 75%, 80%, 85%, 90%, or 95% of the normal range of CH50 levels (42 to 95 U/mL). In some embodiments, the CH50 levels are within 10% to 15%, 15% to 20%, 20% to 25%, 25% to 30%, 30% to 35%, 35% to 40%, 40% to 45%, 50% to 55%, 55% to 60%, 60% to 65%, 65% to 70%, 70% to 75%, or 75% to 80% of the normal range of CH50 levels.

In some embodiments, the present methods have no limitation of use associated with anemia severity, transfusion history, or prior treatment experience. In some embodiments, a subject is at risk of surgery-induced hemolysis. In some embodiments, a subject is at risk of being disqualified from a major surgery.

### Major Surgery

Major surgery, as used herein, refers to any surgical intervention that penetrates and exposes a body cavity; involves removal of an organ or altering the normal anatomy; involves opening the mesenchymal barrier (e.g., pleural cavity, peritoneum, meninges, etc.); is associated extensive tissue dissection or transection; and/or has the potential for inducing permanent anatomic (physical) or physiologic impairments

In some embodiments, the major surgery is cardiac surgery. In some embodiments, the major surgery is gastrointestinal surgery. In some embodiments, the major surgery is orthopedic surgery. In some embodiments, the major surgery is oral surgery. In some embodiments, the major surgery is cranial surgery. In some embodiments, the major surgery is urologic surgery. In some embodiments, the major surgery is organ replacement. In some embodiments, the surgery is an emergency surgery for trauma.

In some embodiments, the major surgery is associated with hypoxia.

In some embodiments, the major surgery is associated with a drop in body temperature. In some embodiments, the major surgery is associated with a drop in body temperature of about 1° C., 2° C., 3° C., 4° C., 5° C., 6° C., 7° C., 8° C., 9° C., 10° C., or more. In some embodiments, the major surgery is associated with a drop in body temperature of about 1° C.-3° C., about 3° C. -5° C., about 5° C. -7° C., or about 7° C. -10° C.

In some embodiments, the major surgery is associated with hemodilution.

In some embodiments, the major surgery is associated with extracorporeal circulation. For example, extracorporeal circulation may be required for cardiopulmonary bypass in the course of a cardiac surgery. In some embodiments, the extracorporeal circulation involves the use of a heart-lung machine.

In some embodiments, the major surgery is major cardiac surgery. In some embodiments, the major cardiac surgery is aortic surgery, aortic valve surgery, arrhythmia surgery, atrial fibrillation surgery, carotid endarterectomy, coronary artery bypass graft (CABG) surgery, heart valve repair or replacement surgery, heart transplant, mitral valve repair, myectomy or myotomy, or, ventricular assist device placement, or transmyocardial revascularization.

In some embodiments, the major cardiac surgery is CABG surgery. In some embodiments, one or more precaution(s) are taken to avoid cooling of the patient. In some embodiments, the CABG surgery involves the use of a heart-lung machine and fluids in the heart-lung machine are kept at 37° C. In some embodiments, no plasma products or coagulation factors are administered during surgery, for example, to avoid replacement of C1q.

### Proximal Classical Complement Pathway Inhibitors

As used herein, the proximal classical complement pathway involves components C1 (which includes C1q, C1r, and C1s proteins), C2, and C4. These components act upstream of C3 in the complement activation cascade. A proximal classical complement pathway inhibitor thus refers to an inhibitor that inhibits (e.g., directly or indirectly inhibits the activity and/or expression) any one of C1q, C1r, C1s, C2, or C4. In some embodiments, the proximal classical complement pathway is a C1q inhibitor. In some embodiments, proximal classical complement pathway inhibitor is a C1r inhibitor. In some embodiments, the proximal classical complement pathway is a C1s inhibitor. In some embodiments, the proximal classical complement pathway inhibitor is a C2 inhibitor. In some embodiments, the proximal classical complement pathway inhibitor is a C4 inhibitor.

A variety of different complement inhibitors may be used in various embodiments of the disclosure. An inhibitor can belong to any of a number of compound classes such as polypeptides (including fusion proteins (e.g., GL-0719 (Gliknik), directed against C1q), cyclic polypeptides, peptidomimetics and cyclic peptidomimetics), small molecule drugs, and nucleic acids (e.g., aptamers and RNAi agents such as short interfering RNAs). In some embodiments, an inhibitor is an antibody.

In some embodiments, the inhibitor specifically binds a proximal classical complement pathway component. In some embodiments, the inhibitor inhibits (e.g., inhibits by at least 20%, at least 30%, at least 40%, at least 50%, at least 60%, at least 70%, at least 80%, or at least 90%, or completely inhibits) an enzymatic activity of a proximal classical complement pathway component. The enzymatic activity may be proteolytic activity, such as the ability to cleave another complement protein.

In some embodiments, the inhibitor is an anti-C1q antibody (e.g., ANX005 (Annexon), ANX007 (Annexon), etc.). Examples of humanized anti-C1q antibodies are disclosed in U.S. Pat. No. 10,316,081, which is hereby incorporated by reference for the antibodies and related compositions that it discloses. In some embodiments, an inhibitor is an anti-C1r antibody. In some embodiments, the inhibitor is an anti-C1s antibody. In some embodiments, the inhibitor is an anti-C2 antibody (e.g., PRO-02 (Prothix)). In some embodiments, the inhibitor is an anti-C4 antibody.

“Antibody” encompasses antibodies or immunoglobulins of any isotype, including but not limited to humanized antibodies and chimeric antibodies. An antibody may be a single-chain antibody (scab) or a single domain antibody (dAb) (e.g., a single domain heavy chain antibody or a single domain light chain antibody: see Holt et al. (2003) Trends Biotechnol. 21:484). The term “antibody” also encompasses fragments of antibodies (antibody fragments) that retain specific binding to an antigen. “Antibody” further includes single-chain variable fragments (scFvs), which are fusion proteins of the variable regions of the heavy (V_{H}) and light chains (V_{L}) of antibodies, connected with a short linker peptide, and diabodies, which are noncovalent dithers of scFv fragments that include the V_{H} and V_{L} connected by a small peptide linker (Zapata et al., Protein Eng. 8(10): 1057-1062 (1995)). Other fusion proteins that comprise an antigen-binding portion of an antibody and a non-antibody protein are also encompassed by the term “antibody.”

“Antibody fragments” comprise a portion of tact antibody, for example, the antigen binding or variable region of the intact antibody. Examples of antibody fragments include an antigen-binding fragment (Fab), Fab′, F(ab′)₂, a variable domain Fv fragment (Fv), an Fd fragment, and an antigen binding fragment of a chimeric antigen receptor.

Papain digestion of antibodies produces two identical antigen-binding fragments, referred to as “Fab” fragments, each with a single antigen-binding site, and a residual “Fc” fragment, a designation reflecting the ability to crystallize readily. Pepsin treatment yields an F(ab′)₂ fragment that has two antigen combining sites and is still capable of cross-linking antigen.

“Fv” is the minimum antibody fragment that contains a complete antigen-recognition and -binding site. This region includes a dimer of one heavy-chain variable domain and one light-chain variable domain in tight, non-covalent association. It is in this configuration that the three CDRs of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

“Fab” fragments contain the constant domain of the light chain and the first constant domain (CH₁) of the heavy chain. Fab fragments differ from Fab′ fragments by the addition of a few residues at the carboxyl terminus of the heavy chain CH; domain including at least one cysteine front the antibody hinge region. Fab′-SH is the designation herein for Fab in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

“scFv” antibody fragments comprise the V_{H} and V_{L} of an antibody, wherein these regions are present in a single polypeptide chain. In some embodiments, the Fv polypeptide further comprises a polypeptide linker between the V_{H} and V_{L} regions, which enables the scFv to form the desired structure for antigen binding. For a review of scFv, see Pluckthun in The Pharmacology of Monoclonal Antibodies, vol. 113, Rosenburg and Moore eds., Springer-Verlag, New York, pp. 269-315 (1994).

“Diabody” refers to a small antibody fragment with two antigen-binding sites, which fragments comprise a VFX connected to a V_{L}, in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, Hollinger et al. Proc. Natl. Aced. Sci. USA 90: 6444-6448 (1993).

An antibody can be monovalent or bivalent. An antibody can be an Ig monomer, which is a “Y-shaped” molecule that consists of four polypeptide chains: two heavy chains and two light chains connected by disulfide bonds.

Antibodies can be detectably labeled, e.g., with a radioisotope, an enzyme that generates a detectable product, and/or a fluorescent protein. Antibodies can be further conjugated to other moieties, such as members of specific binding pairs, e.g., biotin member of biotin-avidin specific binding pair. Antibodies can also be bound to a solid support, including, but not limited to, polystyrene plates and/or beads.

An “isolated” antibody is one that has been identified and separated and/or recovered from a component of its natural environment (i.e., is not naturally occurring). Contaminant components of its natural environment are materials that would interfere with uses (e.g., diagnostic or therapeutic uses) of the antibody, and can include enzymes, hormones, and other proteinaceous or nonproteinaceous solutes. In some embodiments, an antibody is purified (1) to greater than 90%, greater than 95%, or greater than 98% by weight of antibody as determined by the Lowry method, for example, more than 99% by weight, (2) to a degree sufficient to obtain at least 15 residues of N-terminal or internal amino acid sequence by use of a spinning cup sequenator, or (3) to homogeneity by sodium dodecyl sulfate-polyacrylamide gel electrophoresis (SDS-PAGE) under reducing or non-reducing conditions using Coomassie blue or silver stain. isolated antibodies encompass antibodies in situ within recombinant cells, as at least one component of the antibody's natural environment will not be present. In some embodiments, an isolated antibody is prepared by at least one purification step.

A “monoclonal antibody” is an antibody produced by a group of identical cells, all of which were produced from a single cell by repetitive cellular replication. That is, the clone of cells only produces a single antibody species. While a monoclonal antibody can be produced using hybridoma production technology, other production methods known to those skilled in the art can also be used (e.g., antibodies derived from antibody phage display libraries).

A “complementarity determining region (CDR)” is the non-contiguous antigen combining sites found within the variable region of both heavy and light chain polypeptides. CDRs have been described by Lefranc et al. (2003) Developmental and Comparative immunology 27:55; Kabat et al., J. Biol. Chem. 252:6609-6616 (1977); Kabat et al., U S. Dept. of Health and Human Services, “Sequences of proteins of immunological interest” (1991); by Chothia et al., J. Mol. Biol. 196:901-917 (1987); and MacCallum et al., J. Mol. Biol. 262:732-745 (1996), where the definitions include overlapping or subsets of amino acid residues when compared against each other. Nevertheless, application of either definition to refer to a CDR of an antibody or grafted antibodies or variants thereof is intended to be within the scope of the term as defined and used herein.

The terms “LC CDR1,” “LC CDR2,” and “LC CDR3” refer, respectively, to the first, second, and third CDRs in a light chain variable region. As used herein, the terms CDR1″, “FTC CDR2”, and “I-IC CDR3” refer, respectively, to the first, second, and third CDRs in a heavy chain variable region. As used herein, the terms “CDR1”, “CDR2”, and “CDR3” refer, respectively, to the first, second and third CDRs of either chain's variable region.

A “framework” when used in reference to an antibody variable region includes all amino acid residues outside the CDR regions within the variable region of an antibody. A variable region framework is generally a discontinuous amino acid sequence that includes only those amino acids outside of the CDRs. A “framework region” includes each domain of the framework that is separated by the CDRs.

A “humanized antibody” is an antibody comprising portions of antibodies of different origin, wherein at least one portion comprises amino acid sequences of human origin. For example, the humanized antibody can comprise portions derived from an antibody of nonhuman origin with the requisite specificity, such as a mouse, and from antibody sequences of human origin (e.g., chimeric immunoglobulin), joined together chemically by conventional techniques (e.g., synthetic) or prepared as a contiguous polypeptide using genetic engineering techniques (e.g., DNA encoding the protein portions of the chimeric antibody can be expressed to produce a contiguous polypeptide chain). Another example of a humanized antibody is an antibody containing at least one chain comprising a CDR derived from an antibody of nonhuman origin and a framework region derived from a light and/or heavy chain of human origin (e.g., CDR-grafted antibodies with or without framework changes), Chimeric or CDR-grafted single chain antibodies are also encompassed by the term humanized immunoglobulin. See, e.g., Cabilly et at., U.S. Pat. No. 4,816,567; Cabilly et at., European Patent No. 0,125,023 B1; Boss et at., U.S. Pat. No. 4,816,397; Boss et al., European Patent No. 0,120,694 B1; Neuberger, M. S. et al., WO 86/01533; Neuberger, S. et al., European Patent No. 0,194,276 B I; Winter, U.S. Pat. No. 5,225,539; Winter, European Patent No. 0,239,400 B 1; Padlan, E. A. et al., European Patent Application No. 20 0,519,596 A1. See also, Ladner et al., U.S. Pat. No. 4,946,778; Huston, U. S. Pat. No. 5,476,786; and Bird, R, E. et al., Science, 242: 423-4:26 (1988)), regarding single chain antibodies.

In some embodiments, a humanized antibody is produced using synthetic and/or recombinant, nucleic acids to prepare genes (e.g., cDNA.) encoding the desired humanized chain. For example, nucleic acid (e.g., DNA) sequences coding for humanized variable regions can be constructed using PCR mutagenesis methods to alter DNA sequences encoding a human or humanized chain, such as a DNA template from a previously humanized variable region (see e.g., Kamman, M., et al., Nucl. Acids Res., 17: 5404 (1989)); Sato, K., et at., Cancer Research, 53: 851-856 (1993); Daugherty, B. L. et al., Nucleic Acids Res., 19(9): 2471-2476 (1991); and Lewis, A. P. and J. S. Crowe, Gene, 101: 297-302 (1991)). Using these or other suitable methods, variants can also be readily produced. For example, cloned variable regions can be mutagenized, and sequences encoding variants with the desired specificity can be selected (e.g., from a phage library; see e.g., Krebber et al., U.S. Pat. No. 5,514,548; Hoogenboom et al., WO 93/06213, published Apr. 1, 1993).

In some embodiments, a humanized antibody described herein (e.g., an anti-C1s antibody) is a full-length IgG, an Ig monomer, a Fab fragment, a F(ab′)₂ fragment, a Fd fragment, a scFv, a scAb, or a Fv. In some embodiments, a humanized antibody described herein is a full-length IgG. In some embodiments, the heavy chain of any of the humanized anti-C1s antibodies as described herein comprises a heavy chain constant region (CH) or a portion thereof (e.g., CH1, CH2, CH3, or a combination thereof). The heavy chain constant region can of any suitable origin, e.g., human, mouse, rat, or rabbit. In some embodiments, the heavy chain constant region is from a human IgG (a gamma heavy chain), e.g., IgG2, or IgG4.

In some embodiments, mutations can be introduced into the heavy chain constant region of any one of the humanized antibodies (e.g., an anti-C1s antibody) described herein. In some embodiments, one, two or more mutations (e.g., amino acid substitutions) are introduced into the heavy chain constant region (e.g., in a CH2 domain (residues 231-340 of human IgG1) and/or CH3 domain (residues 341-447 of human IgG1) and/or the hinge region, with numbering according to the Kabat numbering system (e.g., the EU index in Kabat)) to increase or decrease the affinity of the antibody for an Fc receptor (e.g., an activated Fc receptor) on the surface of an effector cell. Mutations in the Fe region of an antibody that decrease or increase the affinity of an antibody for an Fe receptor and techniques for introducing such mutations into the Fe receptor or fragment thereof are known to one of skill in the art. Examples of mutations in the Fc receptor of an antibody that can be made to alter the affinity of the antibody for an Fc receptor are described in, e.g., Smith P et al,, (2012) PNAS 109: 6181-6186, U.S. Pat. No. 6,737,056, and International Publication Nos. WO 02/060919; WO 98/23289; and WO 97/34631, which are incorporated herein by reference,

In some embodiments, one, two or more mutations (e.g., amino acid substitutions) are introduced into the hinge region of the heavy chain constant region (CHI domain) such that the number of cysteine residues in the hinge region are altered (e.g., increased or decreased) as described in, e.g., U. S. Pat. No. 5,677,425. The number of cysteine residues in the hinge region of the CH1 domain can be altered to, e.g., facilitate assembly of the light and heavy chains, or to alter (e g , increase or decrease) the stability of the antibody or to facilitate linker conjugation.

In some embodiments, one, two or more amino acid mutations (i.e., substitutions, insertions or deletions) are introduced into an IgG constant domain, or FcRn-binding fragment thereof to alter (e.g., decrease or increase) half-life of the antibody in In some embodiments, the one or more mutations are introduced into an Fc or hinge-Fc domain fragment. See, e.g., International Publication Nos. WO 02/060919; WO 98/23289; and WO 97/34631; and U.S. Pat. Nos. 5,869,046; 6,121,022; 6,277,375; and 6,165,745 for examples of mutations that will alter (e.g., decrease or increase) the half-life of an antibody in vivo,

In some embodiments, the constant region antibody described herein is an IgG1 constant region and comprises a methionine (M) to tyrosine (Y) substitution in position 252, a serine (S) to threonine (T) substitution in position 254, and a threonine (T) to glutamic acid (E) substitution in position 256, numbered according to the EU index as in Kabat. See U. S. Pat. No. 7,658,921, which is incorporated herein by reference. This type of mutant IgG, referred to as “YTE mutant” has been shown to display fourfold increased half-life as compared to wild-type versions of the same antibody (see Dall'Acqua W F et al,, (2006) J

Diol Chem 281: 23514-24). In some embodiments, an antibody comprises an IgG constant domain comprising one, two, three or more amino acid substitutions of amino acid residues at positions 251-257, 285-290, 308-314, 385-389, and 428-436, numbered according to the EU index as in Kabat. Additional mutations that may be introduced to the heavy chain constant region that would increase the half-life of the antibody are known in the art, e.g., the M428L/N434S (EU numbering; M459L/N466S Kabat numbering) mutations as described in Zaleysky et al., Nat. Biotechnol. 2010 February; 28(2): 157-159.

In some embodiments, one, two or more amino acid substitutions are introduced into an IgG constant domain Fe region to alter the effector function(s) of the antibody. The effector ligand to which affinity is altered can be, for example, an Fe receptor or the Cl component of complement. This approach is described in further detail in U. S. Pat Nos. 5,624,821 and 5,648,260. In some embodiments, the deletion or inactivation (through point mutations or other means) of a constant region domain can reduce Fe receptor binding of the circulating antibody thereby increasing tumor localization. See, e.g., U. S. Pat. Nos. 5,585,097 and 8,591,886 for a description of mutations that delete or inactivate the constant domain and thereby increase tumor localization. In some embodiments, at least one amino acid substitutions may be introduced into the Fe region of an antibody described herein to remove potential glycosylation sites on Fc region, which may reduce Pc receptor binding (see, e.g., Shields R L et al., (2001) J Biol Chem 276: 6591-604).

In some embodiments, at least one amino acid in the constant region can he replaced with a different amino acid residue such that the antibody has altered C1q binding and/or reduced or abolished complement dependent cytotoxicity (CDC). This approach is described in further detail in U.S. Pat. No. 6,194,551 (Idusogie et al.). In some embodiments, at least one amino acid residue in the N-terminal region of the CH2 domain of an antibody described herein is altered to thereby alter the ability of the antibody to fix complement. This approach is described further in International Publication No. WO 94/29351, In some embodiments, the Fe region of an antibody described herein is modified to increase the ability of the. antibody to mediate antibody dependent cellular cytotoxicity (ADCC) and/or to increase the affinity of the antibody for an Fcγ receptor. This approach is described further in International Publication No. WO 00/42072.

In some embodiments, to avoid potential complications due to Fab-arm exchange, which is known to occur with native IgG4 mAbs, the antibodies provided herein may comprise a stabilizing ‘Adair’ mutation (Angal S., et al., “A single amino acid substitution abolishes the heterogeneity of chimeric mouse/human (IgG4) antibody,”' Mol Immunol 30, 105-108; 1993), where serine 22.8 (EU numbering; residue 241 Kabat numbering) is converted to proline resulting in an IgG1-like hinge sequence. In some embodiments, to reduce residual antibody-dependent cellular cytotoxicity, a L235E (EU numbering, corresponding to L248E in Kabat numbering) mutation is introduced to the heavy chain constant region, e.g., as described in Benhnia et al., JOURNAL OF VIROLOGY, December 2009, p. 12355-12367.

Anti-C1s Antibodies

In some embodiments, the proximal classical complement pathway inhibitor is a humanized anti-C1s antibody. In some embodiments, the humanized anti-C1s antibody is sutimlimab. In some embodiments, the humanized anti-C1s antibody is COS0098pHv1, COS0098pHv 1-SG1077R, COS0098pHv1-SG1, IPN009VH2VK3-SG4GK, C1_IPN92H0033-SG4GK/IPN93L0024-SK1, C1_IPN92H0288-SG4GK/IPN93L0211-SK1, C1_IPN92H0288-SG4GK/IPN93L0058-SK1, or C1_IPN92H0307-SG4GK/IPN93L0058-SK1. Humanized anti-C1s antibodies are disclosed in International Publication No. WO 2020/230834, the contents of which are incorporated by reference for the antibodies and related compositions that it discloses.

In some embodiments, a humanized anti-C1s antibody comprises a heavy chain complementarity determining region 1 (HC CDR1) comprising the amino acid sequence of NYAMS (SEQ ID NO: 5). In some embodiments, a humanized anti-C1s antibody comprises a heavy chain complementarily determining region 2 (HC CDR2) comprising the amino acid sequence of TISSGGSHTYYLDSVKG (SEQ ID NO: 6). In some embodiments, a humanized anti-C1s antibody comprises a heavy chain complementarity determining region 1 (HC CDR3) comprising the amino acid sequence of LFTGYAMDY (SEQ ID NO: 7). In some embodiments, a humanized anti-C1s antibody comprises an HC CDR1 comprising the amino acid sequence of SEQ ID NO: 5, an HC CDR2 comprising the amino acid sequence of SEQ ID NO: 6, and an HC CDR3 comprising the amino acid sequence of SEQ ID NO: 7.

In some embodiments, a humanized anti-C1s antibody comprises a light chain complementarity determining region 1 (LC CDR1) comprising the amino acid sequence of TASSSVSSSYLH (SEQ ID NO: 8). In some embodiments, a humanized anti-C1s antibody comprises a light chain complementarity determining region 1 (LC CDR1) comprising the amino acid sequence of STSNLAS (SEQ ID NO: 9). In some embodiments, a humanized anti-C1s antibody comprises a light chain complementarity determining region 1 (LC CDR1) comprising the amino acid sequence of HQYYRLPPIT (SEQ ID NO: 10). In some embodiments, a humanized anti-C1s antibody comprises an LC CDR1 comprising the amino acid sequence of SEQ ID NO: 8, an LC CDR2 comprising the amino acid sequence of SEQ ID NO: 9, and an LC CDR3 comprising the amino acid sequence of SEQ ID NO: 10.

In some embodiments, a humanized anti-C1s antibody comprises an HC CDR1 comprising the amino acid sequence of SEQ ID NO: 5, an HC CDR2 comprising the amino acid sequence of SEQ ID NO: 6, an HC CDR3 comprising the amino acid sequence of SEQ ID NO: 7, an LC CDR1 comprising the amino acid sequence of SEQ ID NO: 8, an LC CDR2 comprising the amino acid sequence of SEQ ID NO: 9, and an LC CDR3 comprising the amino acid sequence of SEQ ID NO: 10.

In some embodiments, a humanized anti-C1s antibody comprises a heavy chain variable region (VH) comprising the amino acid sequence of

(SEQ ID NO: 3)
EVQLVESGGGLVKPGGSLRLSCAASGFTFSNYAMSWVRQAPGKGLEWVA
TISSGGSHTYYLDSVKGRFTISRDNSKNTLYLQMNSLRAEDTALYYCAR
LFTGYAMDYWGQGTLVTVSS.

In some embodiments, a humanized anti-C1s antibody comprises a light chain variable region (VL) comprising the amino acid sequence of

(SEQ ID NO: 4)
QIVLTQSPATLSLSPGERATMSCTASSSVSSSYLHWYQQKPGKAPKLWI
YSTSNLASGVPSRFSGSGSGTDYTLTISSLQPEDFATYYCHQYYRLPPI
TFGQGTKLEIK.

In some embodiments, a humanized anti-C1s antibody comprises a VH comprising the amino acid sequence of SEQ ID NO: 3 and a VL comprising the amino acid sequence of SEQ ID NO: 4.

In some embodiments, a humanized anti-C1s antibody comprises a heavy chain (HC) comprising the amino acid sequence of

(SEQ ID NO: 1)
EVQLVESGGGLVKPGGSLRLSCAASGFTFSNYAMSWVRQAPGKGLEWVA
TISSGGSHTYYLDSVKGRFTISRDNSKNTLYLQMNSLRAEDTALYYCAR
LFTGYAMDYWGQGTLVTVSSASTKGPSVFPLAPCSRSTSESTAALGCLV
KDYFPEPVTVSWNSGALTSGVHTFPAVLQSSGLYSLSSVVTVPSSSLGT
KTYTCNVDHKPSNTKVDKRVESKYGPPCPPCPAPEFEGGPSVFLFPPKP
KDTLMISRTPEVTCVVVDVSQEDPEVQFNWYVDGVEVHNAKTKPREEQF
NSTYRVVSVLTVLHQDWLNGKEYKCKVSNKGLPSSIEKTISKAKGQPRE
PQVYTLPPSQEEMTKNQVSLTCLVKGFYPSDIAVEWESNGQPENNYKTT
PPVLDSDGSFFLYSRLTVDKSRWQEGNVFSCSVMHEALHNHYTQKSLSL
SLGK.

In some embodiments, a humanized anti-C1s antibody comprises a light chain comprising the amino acid sequence of

(SEQ ID NO: 2)
QIVLTQSPATLSLSPGERATMSCTASSSVSSSYLHWYQQKPGKAPKLWI
YSTSNLASGVPSRFSGSGSGTDYTLTISSLQPEDFATYYCHQYYRLPPI
TFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYAC
EVTHQGLSSPVTKSFNRGEC.

In some embodiments, a humanized anti-C1s antibody comprises a HC comprising the amino acid sequence of SEQ ID NO: 1 and a LC comprising the amino acid sequence of SEQ ID NO: 2.

In some embodiments, a humanized anti-C1s antibody comprises an HC CDR1 comprising an amino acid sequence containing no more than 3 amino acid variations (e.g., no more than 3, 2, or 1 amino acid variation(s)) relative to the HC CDR1 amino acid sequence of SEQ ID NO: 5. In some embodiments, a humanized anti-C1s antibody comprises an HC CDR2 comprising an amino acid sequence containing no more than 3 amino acid variations (e.g., no more than 3, 2, or 1 amino acid variation(s)) relative to the HC CDR2 amino acid sequence of SEQ ID NO: 6. In some embodiments, a humanized anti-C1s antibody comprises an HC CDR3 comprising an amino acid sequence containing no more than 3 amino acid variations (e.g., no more than 3, 2, or I amino acid variation(s)) relative to the HC CDR3 amino acid sequence of SEQ ID NO: 7. In some embodiments, affinity maturation may be used to identify CDR variations that preserve binding specificity.

In some embodiments, a humanized anti-C1s antibody comprises an LC CDR1 comprising an amino acid sequence containing no more than 3 amino acid variations (e.g., no more than 3, 2, or 1 amino acid variation(s)) relative to the LC CDR1 amino acid sequence of SEQ ID NO: 8. In some embodiments, a humanized anti-C1s antibody comprises an LC CDR2 comprising an amino acid sequence containing no more than 3 amino acid variations (e.g., no more than 3, 2, or 1 amino acid variation(s)) relative to the LC CDR2 amino acid sequence of SEQ ID NO: 9. In some embodiments, a humanized anti-C1s antibody comprises an LC CDR3 comprising an amino acid sequence containing no more than 3 amino acid variations (e.g., no more than 3, 2, or 1 amino acid variation(s)) relative to the LC CDR3 amino acid sequence of SEQ ID NO: 10.

In some embodiments, a humanized anti-C1s antibody comprises a VH comprising an amino acid sequence containing no more than 20 amino acid variations (e.g., no more, than 20, 19, 18, 17, 16, 15, 14, 13, 12, 11, 10, 9, 8, 7, 6, 5, 4, 3, 2, or I amino acid variation(s)) relative to the VII amino acid sequence of SEQ ID NO: 3.

In some embodiments, a humanized and-C1s antibody comprises a VI, comprising an amino acid sequence containing no more than 20 amino acid variations (e.g., no more than 20, 19, 18, 17, 16, 15, 14, 13, 12, 11, 10, 9, 8, 7, 6, 5, 4, 3, 2, or 1 amino acid variation(s)) relative to the VL amino acid sequence of SEQ ID NO: 4.

In some embodiments, a humanized anti-C1s antibody comprises a VH comprising an HC CDR1 comprising the amino acid sequence of SEQ ID NO: 5, an HC CDR2 comprising the amino acid sequence of SEQ ID NO: 6, an HC CDR3 comprising the amino acid sequence of SEQ ID NO: 7, and comprises framework regions that contain no more than 20 amino acid variations (e.g., no more than 20, 19, 18, 17, 16, 15, 14, 13, 12, 11, 10, 9, 8, 7, 6, 5, 4, 3, 2, or I amino acid variation(s)) relative to the VH sequence of SEQ ID NO: 3.

In some embodiments, a humanized anti-C1s antibody comprises a VL comprising an LC CDR1 comprising the amino acid sequence of SEQ ID NO: 8, an LC CDR2 comprising the amino acid sequence of SEQ ID NO: 9, an LC CDR3 comprising the amino acid sequence of SEQ ID NO: 10, and comprises framework regions that contain no more than 20 amino acid variations (e.g., no more, than 20, 19, 18, 17, 16, 15, 14, 13, 12, 11, 10, 9, 8, 7, 6, 5, 4, 3, 2, or 1 amino acid variation(s)) relative to the VL sequence of SEQ ID NO: 4.

In some embodiments, a humanized anti-C1s antibody comprises (a) a VH comprising an HC CDR1 comprising the amino acid sequence of SEQ ID NO: 5, an HC CDR2 comprising the amino acid sequence of SEQ ID NO: 6, an FIC CDR3 comprising the amino acid sequence of SEQ ID NO: 7, and comprises framework regions that contain no more than 20 amino acid variations (e.g., no more than 20, 19, 18, 17, 16, 15, 14, 13, 12, 11, 10, 9, 8, 7, 6, 5, 4, 3, 2, or 1 amino acid variation(s)) relative to the VII sequence of SEQ ID NO: 3, and (b) a VL comprising an LC CDR1 comprising the amino acid sequence of SEQ ID NO: 8, an LC CDR2 comprising the amino acid sequence of SEQ ID NO: 9, an LC CDR3 comprising the amino acid sequence of SEQ ID NO: 10, and comprises framework regions that contain no more, than 20 amino acid variations (e.g., no more than 20, 19, 18, 17, 16, 15, 14, 13, 12, 11, 10, 9, 8, 7, 6, 5, 4, 3, 2, or 1 amino acid variation(s)) relative to the VL sequence of SEQ ID NO: 4.

In some embodiments, a humanized anti-C1s antibody comprises a VH comprising an amino acid sequence having at least 80% (e.g., 80%, 85%, 90%, 95%, 96%, 97%, 98%, or 99%) identity to the VH amino acid sequence of SEQ ID NO. 3.

In some embodiments, a humanized anti-C1s antibody comprises a VL comprising an amino acid sequence having at least 80% (e.g., 80%, 85%, 90%, 95%, 96%, 97%, 98%, or 99%) identity to the VL amino acid sequence of SEQ ID NO: 4.

In some embodiments, a humanized anti-C1s antibody comprises a VH comprising an HC CDR1 comprising the amino acid sequence of SEQ ID NO: 5, an HC CDR2 comprising the amino acid sequence of SEQ ID NO: 6, an HC CDR3 comprising the amino acid sequence of SEQ ID NO: 7 and comprises framework regions that have at least 80% (e.g., 80%, 85%, 90%, 95%, 96%, 97%, 98%, or 99%) identity to the framework regions of the VI-I sequence of SEQ ID NO: 3.

In some embodiments, a humanized anti-C1s antibody comprises a VL comprising an LC CDR1 comprising the amino acid sequence of SEQ LD NO: 8, an L.C. CDR2 comprising the amino acid sequence of SEQ ID NO: 9, an LC CDR3 comprising the amino acid sequence of SEQ ID NO: 10 and comprises framework regions that have at least 80% (e.g., 80%, 85%, 90%, 95%, 96%, 97%, 98%, or 99%3 identity to the framework regions of the VL sequence of SEQ ID NO: 4.

In some embodiments, a humanized anti-C1s antibody comprises (a) a VH comprising an HC CDR1 comprising the amino acid sequence of SEQ ID NO: 5, an HC CDR2 comprising the amino acid sequence of SEQ ID NO: 6, an HC CDR3 comprising the amino acid sequence of SEQ ID NO: 7, and comprises framework regions that have at least 80% (e.g., 80%, 85%, 90%, 95%, 96%, 97%, 98%, or 99%) identity to the framework regions of the VH sequence of SEQ ID NO: 3, and (b) a VL comprising an LC CDR1. comprising the amino acid sequence of SEQ ID NO: 8, an LC CDR2 comprising the amino acid sequence of SEQ ID NO: 9, an LC CDR3 comprising the amino acid sequence of SEQ ID NO: 10, and comprises framework regions that have at least 80% (e.g., 80%, 85%, 90%, 95%, 96%, 97%, 98%, or 99%) identity to the framework regions of the VL sequence of SEQ ID NO: 4.

In some embodiments, a humanized anti-C1s antibody comprises a heavy chain complementarity determining region 1 (HC CDR1) comprising the amino acid sequence of (SEQ ID NO: 15). In some embodiments, a humanized anti-C1s antibody comprises a heavy chain complementarily determining region 2 (HC CDR2) comprising the amino acid sequence of RIDPADGHTKYAPKFQV (SEQ ID NO: 16). In some embodiments, a humanized anti-C1s antibody comprises a heavy chain complementarity determining region 1 (HC CDR3) comprising the amino acid sequence of YGYGREVFDY (SEQ ID NO: 17). In some embodiments, a humanized anti-C1s antibody comprises an HC CDR1 comprising the amino acid sequence of SEQ ID NO: 15, an FIC CDR2 comprising the amino acid sequence of SEQ ID NO: 16, and an TIC CDR3 comprising the amino acid sequence of SEQ ID NO: 17.

In some embodiments, a humanized anti-C1s antibody comprises a light chain complementarity determining region 1 (LC CDR1) comprising the amino acid sequence of KASQSVDYDGDSYMN (SEQ ID NO: 18). In some embodiments, a humanized anti-C1s antibody comprises a light chain complementarity determining region 1 (LC CDR1) comprising the amino acid sequence of DASNLES (SEQ ID NO: 19). In some embodiments, a humanized anti-C1s antibody comprises a light chain complementarity determining region 1 (LC CDR1) comprising the amino acid sequence of QQSNEDVWF (SEQ ID NO: 20). In some embodiments, a humanized anti-C1s antibody comprises an LC CDR1 comprising the amino acid sequence of SEQ ID NO: 18, an LC CDR2 comprising the amino acid sequence of SEQ ID NO: 19, and an LC CDR3 comprising the amino acid sequence of SEQ ID NO: 20.

In some embodiments, a humanized anti-C1s antibody comprises an FIC CDR1 comprising the amino acid sequence of SEQ ID NO: 15, an I-IC CDR2 comprising the amino acid sequence of SEQ ID NO: 16, an HC CDR3 comprising the amino acid sequence of SEQ ID NO: 17, an LC CDR1 that comprises the amino acid sequence of SEQ ID NO: 18, an LC CDR2 comprising the amino acid sequence of SEQ ID NO: 19, and an LC CDR3 comprising the amino acid sequence of SEQ ID NO: 20.

In some embodiments, a humanized anti-C1s antibody comprises a heavy chain variable region (VH) comprising the amino acid sequence of

(SEQ ID NO: 13)
QVQLVQSGAEVKKPGASVKLSCTASGFNIKDDYIHWVKQAPGQGLEWIG
RIDPADGHTKYAPKFQVKVTITADTSTSTAYLELSSLRSEDTAVYYCAR
YGYGREVFDYWGQGTTVTVSS.

In some embodiments, a humanized anti-C1s antibody comprises a light chain variable region (VL) comprising the amino acid sequence of

(SEQ ID NO: 14)
DIVLTQSPDSLAVSLGERATISCKASQSVDYDGDSYMNWYQQKPGQPPK
ILIYDASNLESGIPARFSGSGSGTDFTLTISSLEPEDFAIYYCQQSNED
PWTFGGGTKVEIK.

In some embodiments, a humanized anti-C1s antibody comprises a VH comprising the amino acid sequence of SEQ ID NO: 13 and a VL comprising the amino acid sequence of SEQ ID NO: 14.

In some embodiments, a humanized anti-C1s antibody comprises a heavy chain (HC) comprising the amino acid sequence of

(SEQ ID NO: 11)
QVQLVQSGAEVKKPGASVKLSCTASGFNIKDDYIHWVKQAPGQGLEWIG
RIDPADGHTKYAPKFQVKVTITADTSTSTAYLELSSLRSEDTAVYYCAR
YGYGREVFDYWGQGTTVTVSSASTKGPSVFPLAPCSRSTSESTAALGCL
VKDYFPEPVTVSWNSGALTSGVHTFPAVLQSSGLYSLSSVVTVPSSSLG
TKTYTCNVDHKPSNTKVDKRVESKYGPPCPPCPAPEFEGGPSVFLFPPK
PKDTLMISRTPEVTCVVVDVSQEDPEVQFNWYVDGVEVHNAKTKPREEQ
FNSTYRVVSVLTVLHQDWLNGKEYKCKVSNKGLPSSIEKTISKAKGQPR
EPQVYTLPPSQEEMTKNQVSLTCLVKGFYPSDIAVEWESNGQPENNYKT
TPPVLDSDGSFFLYSRLTVDKSRWQEGNVFSCSVLHEALHSHYTQKSLS
LSLGK.

In some embodiments, a humanized anti-C1s antibody comprises a light chain (LC) comprising the amino acid sequence of

(SEQ ID NO: 12)
DIVLTQSPDSLAVSLGERATISCKASQSVDYDGDSYMNWYQQKPGQPPK
ILIYDASNLESGIPARFSGSGSGTDFTLTISSLEPEDFAIYYCQQSNED
PWTFGGGTKVEIKRTVAAPSVFIFPPSDEQLKSGTASVVCLLNNFYPRE
AKVQWKVDNALQSGNSQESVTEQDSKDSTYSLSSTLTLSKADYEKHKVY
ACEVTHQGLSSPVTKSENRGEC.

In some embodiments, a humanized anti-C1s antibody comprises a HC comprising the amino acid sequence of SEQ ID NO: 11 and a LC comprising the amino acid sequence of SEQ ID NO: 12.

In some embodiments, a humanized anti-C1s antibody comprises an HC CDR1 comprising an amino acid sequence containing no more than 3 amino acid variations (e.g., no more than 3, 2, or 1 amino acid variation(s)) relative to the HC CDR1 amino acid sequence of SEQ ID NO: 15. In some embodiments, a humanized anti-C1s antibody comprises an HC CDR2 comprising an amino acid sequence containing no more than 3 amino acid variations (e.g., no more than 3, 2, or l amino acid variation(s)) relative to the FIC CDR2 amino acid sequence of SEQ ID NO: 16. In some embodiments, a humanized anti-C1s antibody comprises an HC CDR3 comprising an amino acid sequence containing no more than 3 amino acid variations (e.g., no more than 3, 2, or 1 amino acid variation(s)) relative to the HC CDR3 amino acid sequence of SEQ ID NO: 7.

In some embodiments, a humanized anti-C1s antibody comprises an LC CDR1 comprising an amino acid sequence containing no more than 3 amino acid variations (e.g., no more than 3, 2, or 1 amino acid variation(s)) relative to the LC CDR1 amino acid sequence of SEQ ID NO: 18. In some embodiments, a humanized anti-C1s antibody comprises an LC CDR2 comprising an amino acid sequence containing no more than 3 amino acid variations (e.g., no more than 3, 2, or 1 amino acid variation(s)) relative to the LC CDR2 amino acid sequence of SEQ ID NO: 19. In some embodiments, a humanized anti-C1s antibody comprises an LC CDR3 comprising an amino acid sequence containing no more than 3 amino acid variations (e.g., no more than 3, 2, or 1 amino acid variation(s)) relative to the LC CDR3 amino acid sequence of SEQ ID NO: 20.

In some embodiments, a humanized anti-C1s antibody comprises a VH comprising an amino acid sequence containing no more than 20 amino acid variations (e.g., no more than 20, 19, 18, 17, 16, 15, 14, 13, 12, 11, 10, 9, 8, 7, 6, 5, 4, 3, 2, or 1 amino acid variation(s)) relative to the VH amino acid sequence of SEQ ID NO: 13.

In some embodiments, a humanized anti-C1s antibody comprises a VL comprising an amino acid sequence containing no more than 20 amino acid variations (e.g., no more than 20, 19, 18, 17, 16, 15, 14, 13, 12, 11, 10, 9, 8, 7, 6. 5, 4, 3, 2, or 1 amino acid variation(s)) relative to the VL amino acid sequence of SEQ ID NO: 14.

In some embodiments, a humanized anti-C1s antibody comprises a VH comprising an HC CDR1 comprising the amino acid sequence of SEQ ID NO: 15, an FIC CDR2 comprising the amino acid sequence of SEQ ID NO: 16, an HC CDR3 comprising the amino acid sequence of SEQ ID NO: 17, and comprises framework regions that contain no more than 20 amino acid variations (e.g., no more, than 20, 19, 18, 17, 16, 15, 14, 13, 12, 11, 10, 9, 8, 7, 6, 5, 4, 3, 2, or 1 amino acid variation(s)) relative to the VH sequence of SEQ ID NO: 13.

In some embodiments, a humanized anti-C1s antibody comprises a VL comprising an LC CDR1 comprising the amino acid sequence of SEQ ID NO: 18, an LC CDR2 comprising the amino acid sequence of SEQ ID NO: 19, an LC CDR3 comprising the amino acid sequence of SEQ ID NO: 20, and comprises framework regions that contain no more than 20 amino acid variations (e.g., no more than 20, 19, 18, 17, 16, 15, 14, 13, 12, 11, 10, 9, 8, 7, 6, 5, 4, 3, 2, or 1 amino acid variation(s)) relative to the VL sequence of SEQ ID NO: 14.

In some embodiments, a humanized anti-C1s antibody comprises (a) a VH comprising an HC CDR1 comprising the amino acid sequence of SEQ ID NO: 15, an HC CDR2 comprising the amino acid sequence of SEQ ID NO: 16, an FIC CDR3 comprising the amino acid sequence of SEQ ID NO: 17, and comprises framework regions that contain no more than 20 amino acid variations (e.g., no more than 20, 19, 18, 17, 16, 15, 14, 13, 12, 11, 10, 9, 8, 7, 6, 5, 4, 3, 2, or 1 amino acid variation(s)) relative to the VH sequence of SEQ ID NO: 13, and (b) a VL comprising an LC CDR1 comprising the amino acid sequence of SEQ NO: 18, an LC CDR2 comprising the amino acid sequence of SEQ ID NO: 19, an LC CDR3 comprising the amino acid sequence of SEQ ID NO: 20, and comprises framework regions that contain no more than 20 amino acid variations (e.g., no more than 20, 19, 18, 17, 16, 15, 14, 13, 12, 11, 10,9, 8, 7, 6, 5, 4, 3, 2, or 1 amino acid variation(s)) relative to the VL sequence of SEQ ID NO: 14.

In some embodiments, a humanized anti-C1s antibody comprises a VH comprising an amino acid sequence having at least 80% (e.g., 80%, 85%, 90%, 95%, 96%, 97%, 98%, or 99%) identity to the VH amino acid sequence of SEQ ID NO: 13.

In some embodiments, a humanized anti-C1s antibody comprises a VL comprising an amino acid sequence having at least 80% (e.g., 80%, 85%, 90%, 95%, 96%, 97%, 98%, or 99%) identity to the VL amino acid sequence of SEQ ID NO: 14.

In some embodiments, a humanized anti-C1s antibody comprises a VH comprising an HC CDR1 comprising the amino acid sequence of SEQ ID NO: 15, an HC CDR2 comprising the amino acid sequence of SEQ ID NO: 16, an HC CDR3 comprising the amino acid sequence of SEQ ID NO: 17 and comprises framework regions that have at least 80% (e.g., 80%, 85%, 90%, 95%, 96%, 97%, 98%, or 99%) identity to the framework regions of the VII sequence of SEQ ID NO: 13.

In some embodiments, a humanized anti-C1s antibody comprises a VL comprising an HC CDR1 comprising the amino acid sequence of SEQ ID NO: 18, an LC CDR2 comprising the amino acid sequence of SEQ ID NO: 19, an LC CDR3 comprising the amino acid sequence of SEQ ID NO: 20 and comprises framework regions that have at least 80% (e.g., 80%, 85%, 90%, 95%, 96%, 97%, 98%, or 99%) identity to the framework regions of the VL sequence of SEQ ID NO: 14.

In some embodiments, a humanized anti-C1s antibody comprises (a) a VH comprising an HC CDR1 comprising the amino acid sequence of SEQ ID NO: 15, an HC CDR2 comprising the amino acid sequence of SEQ ID NO: 16, an HC CDR3 comprising the amino acid sequence of SEQ ID NO: 17, and comprises framework regions that have at least 80% (e.g., 80%, 85%, 90%, 95%, 96%, 97%, 98%, or 99%) identity to the framework regions of the VH sequence of SEQ ID NO: 13, and (b) a VL comprising an LC CDR1 comprising the amino acid sequence of SEQ ID NO: 18, an LC CDR2 comprising the amino acid sequence of SEQ ID NO: 19, an LC CDR3 comprising the amino acid sequence of SEQ ID NO: 20, and comprises framework regions that have at least 80% (e.g., 80%, 85%, 90%, 95%, 96%, 97%, 98%, or 99%) identity to the framework regions of the VL sequence of SEQ ID NO: 14.

In some embodiments, the heavy chain constant region in any one of the humanized anti-C1s antibodies described herein is an IgG4 constant region, or a variant there of. Examples of IgG4 constant regions and variants are provided in Table 1.

TABLE 1
Examples of Heavy Chain Constant Regions

| Heavy Chain | |
|---|---|
| Constant | |
| Region | Amino Acid Sequence |
| IgG4 constant | ASTKGPSVFPLAPCSRSTSESTAALGCLVKDYFPEPVTVSWNSGALTSGVHTFPAVLQSSG |
| region WT | LYSLSSVVTVPSSSLGTKTYTCNVDHKPSNTKVDKRVESKYGPPCPSCPAPEFLGGPSVFL |
| | FPPKPKDTLMISRTPEVTCVVVDVSQEDPEVQFNWYVDGVEVHNAKTKPREEQFNSTYRVV |
| | SVLTVLHQDWLNGKEYKCKVSNKGLPSSIEKTISKAKGQPREPQVYTLPPSQEEMTKNQVS |
| | LTCLVKGFYPSDIAVEWESNGQPENNYKTTPPVLDSDGSFFLYSRLTVDKSRWQEGNVFSC |
| | SVMHEALHNHYTQKSLSLSLGK (SEQ ID NO: 21) |
| IgG4 constant | ASTKGPSVFPLAPCSRSTSESTAALGCLVKDYFPEPVTVSWNSGALTSGVHTFPAVLQSSG |
| region variant 1 | LYSLSSVVTVPSSSLGTKTYTCNVDHKPSNTKVDKRVESKYGPPCPPCPAPEFEGGPSVFL |
| | FPPKPKDTLMISRTPEVTCVVVDVSQEDPEVQFNWYVDGVEVHNAKTKPREEQFNSTYRVV |
| | SVLTVLHQDWLNGKEYKCKVSNKGLPSSIEKTISKAKGQPREPQVYTLPPSQEEMTKNQVS |
| | LTCLVKGFYPSDIAVEWESNGQPENNYKTTPPVLDSDGSFFLYSRLTVDKSRWQEGNVFSC |
| | SVMHEALHNHYTQKSLSLSLGK (SEQ ID NO: 22) |
| IgG4 constant | ASTKGPSVFPLAPCSRSTSESTAALGCLVKDYFPEPVTVSWNSGALTSGVHTFPAVLQSSG |
| region variant 2 | LYSLSSVVTVPSSSLGTKTYTCNVDHKPSNTKVDKRVESKYGPPCPPCPAPEFEGGPSVFL |
| | FPPKPKDTLMISRTPEVTCVVVDVSQEDPEVQFNWYVDGVEVHNAKTKPREEQFNSTYRVV |
| | SVLTVLHQDWLNGKEYKCKVSNKGLPSSIEKTISKAKGQPREPQVYTLPPSQEEMTKNQVS |
| | LTCLVKGFYPSDIAVEWESNGQPENNYKTTPPVLDSDGSFFLYSRLTVDKSRWQEGNVESC |
| | SVLHEALHSHYTQKSLSLSLGK (SEQ ID NO: 23) |

In some embodiments, the light chain of any of the humanized anti-C1s antibodies described herein may further comprise a light chain constant region (C_{L}). In some examples, the C_{L} is a kappa light chain. In other examples, the C_{L} is a lambda light chain. In some embodiments, the C_{L} is a kappa light chain, the sequence of which is provided below:

(SEQ ID NO: 24)
RTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPV
IKSENRGEC

Other antibody heavy and light chain constant regions are well known in the art, e.g., those provided in the IMGT database (imgt.org) or at vbase2.org/vbstat.php, both of which are incorporated by reference herein.

### Compositions

A proximal classical complement pathway inhibitor (e.g., an anti-C1s antibody) is generally present in a composition, e.g., a pharmaceutical composition.

A composition comprising an inhibitor (e.g., an anti-C1s antibody), in some embodiments, comprises one or more of a salt, e.g., NaCl, MgCl₂, KCl, MgSO₄, etc.; a buffering agent, e.g., a Tris buffer, N-(2-Hydroxyethyl)piperazine-N-(2-ethanesulfonic acid) (HEPES), 2-(N-Morpholino)ethanesulthnic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.; a solubilizing agent; a detergent, e.g., a non-ionic detergent such as Tween-20, etc.; a protease inhibitor; and/or glycerol.

An inhibitor (e.g., an anti-C1s antibody) May be administered to a subject using any convenient means capable of resulting in the desired therapeutic effect. Thus, the inhibitor (e.g., an anti-C1s antibody) may be incorporated into a variety of formulations for therapeutic administration. For example, an inhibitor (e.g., an anti-C1s antibody) may be formulated into pharmaceutical compositions by combination with appropriate, pharmaceutically acceptable carriers, pharmaceutically acceptable diluents, or other pharmaceutically acceptable excipients and can be formulated into preparations in solid, semi solid, liquid or gaseous forms, such as tablets, capsules, powders, granules, ointments, solutions, suppositories, injections, inhalants and aerosols. In some embodiments, a pharmaceutical composition comprises an inhibitor (e.g., an anti-C1s antibody) and a pharmaceutically acceptable excipient.

In pharmaceutical dosage forms, an inhibitor (e.g., an anti-C1s antibody) can be administered in the form of their pharmaceutically acceptable salts, or they can also be used alone or in appropriate association, as well as in combination, with other pharmaceutically active compounds.

For oral preparations, an inhibitor (e.g., an anti-C1s antibody) can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

An inhibitor (e.g., an anti-C1s antibody) can be formulated into preparations for injection by dissolving, suspending or emulsifying the antibody in an aqueous or nonaqueous solvent, such as vegetable or other similar oils, propylene glycol, synthetic aliphatic acid glycerides, injectable organic esters (e.g., ethyl oleate), esters of higher aliphatic acids or propylene glycol; and if desired, with conventional additives such as solubilizers, isotonic agents, suspending agents, emulsifying agents, stabilizers and preservatives. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Furthermore, the pharmaceutical composition of the present disclosure can comprise further agents such as dopamine or psychopharmacologic drugs, depending on the intended use of the pharmaceutical composition.

Pharmaceutical compositions comprising an inhibitor (e.g., an anti-C1s antibody)are prepared by mixing a subject inhibitor having the desired degree of purity with optional physiologically acceptable carriers, other excipients, stabilizers, surfactants, buffers and/or tonicity agents. Acceptable carriers, other excipients and/or stabilizers are nontoxic to recipients at the dosages and concentrations employed, and include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride, or combinations thereof); amino acids such as arginine, glycine, ornithine, lysine, histidine, glutamic acid, aspartic acid, isoleucine, leucine, alanine, phenylalanine, tyrosine, tryptophan, methionine, serine, proline and combinations thereof; monosaccharides, disaccharides and other carbohydrates; low molecular weight (less than about 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine, N-methylglucosamine, galactosamine, and neuraminic acid; and/or non-ionic surfactants such as Tween, Brij Pluronics, Triton-X, or polyethylene glycol (PEG).

The pharmaceutical composition can be in a liquid form, a lyophilized form or a liquid form reconstituted from a lyophilized form, wherein the lyophilized preparation is to be reconstituted with a sterile solution prior to administration. The standard procedure for reconstituting a lyophilized composition is to add hack a volume of pure water (typically equivalent to the volume removed during lyophilization); however solutions comprising antibacterial agents can be used for the production of pharmaceutical compositions for parenteral administration; see also Chen (1992) Drug Dev Ind Pharm 18, 1311-54.

Exemplary inhibitor (e.g., anti-C1s antibody) concentrations in a pharmaceutical composition suitable for use in a method of the present disclosure can range from about 1 mg/mL to about 200 ma/mL, or from about 50 mg/mL to about 200 mg/mL, or from about 150 mg/mL to about 200 mg/mL. In some aspects, the inhibitor anti-C1s antibody) concentration is from about 10 μg/mL to about 60 mg/mL, from about 12 mg/mL to about 58 10 mg/mL, from about 14 mg/mL, to about 56 mg/ml, from about 16 mg/ml, to about 54 mg/mL, from about 17 mg/mL to about 52 μg/mL, or from about 18 mg/mL to about 50 mg/mL. In some aspects, the inhibitor (e.g., anti-C1s antibody) concentration is 18 mg/mL. In some aspects, the inhibitor (e.g., anti-C1s antibody) concentration is 50 mg/mL.

An aqueous formulation of an inhibitor (e.g., an anti-C1s antibody) can be prepared in a pH-buffered solution, e.g., at pH ranging from about 4.0 to about 7.0, or from about 5.0 to about 6.0, or alternatively about 5.5. Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation,

A tonicity agent can be included in the inhibitor (e.g., an anti-C1s antibody) formulation to modulate the tonicity of the formulation. Exemplary tonicity agents include sodium chloride, potassium chloride, glycerin and any component from the group of amino acids, sugars as well as combinations thereof. In some embodiments, the aqueous formulation is isotonic, although hypertonic or hypotonic solutions can be suitable. The term “isotonic” denotes a solution having the same tonicity as some other solution with which it is compared, such as a physiological salt solution or serum. Tonicity agents can be used in an amount of about 5 mM to about 350 mM, e.g., in an amount of 100 mM to 350 nM.

A surfactant can also be added to the inhibitor (e.g., an anti-C1s antibody) formulation to reduce aggregation of the formulated inhibitor and/or minimize the formation of particulates in the formulation and/or reduce adsorption. Exemplary surfactants include polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), and sodium dodecyl sulfate (SDS). Examples of suitable polyoxyethylenesorbitan-fatty acid esters are polysorbate 20, (sold under the trademark Tween 20™) and polysorbate 80 (sold under the trademark TWEEN 80™). Examples of suitable polyethylene-polypropylene copolymers are those sold under the names PLURONIC® F68 or POLOXAMER ISS™. Examples of suitable Polyoxyethylene alkyl ethers are those sold under the trademark BRIJ™. Exemplary concentrations of surfactant can range from about 0.001% to about 1% w/v.

A lyoprotectant can also be added in order to protect the labile active ingredient (e.g. a protein) against destabilizing conditions during the lyophilization process. For example, known lyoprotectants include sugars (including glucose and sucrose); polyols (including mannitol, sorbitol and glycerol); and amino acids (including alanine, glycine and glutamic acid). Lyoprotectants can be included in an amount of about 10 mM to 500 nM.

In some embodiments, a suitable formulation includes an inhibitor (e.g., an anti-C1s antibody), and one or more of the above-identified agents (e.g., a surfactant, a buffer, a stabilizer, a tonicity agent) and is essentially free of one or more preservatives, such as ethanol, benzyl alcohol, phenol, m-cresol, p-chlor-m-cresol, methyl or propyl parabens, benzalkonium chloride, and combinations thereof. In other embodiments, a preservative is included in the formulation, e.g., at concentrations ranging from about 0.001 to about 2% (w/v).

For example, a suitable formulation can be a liquid or lyophilized formulation suitable for parenteral administration and can comprise: about 1 mg/mL to about 200 mg/mL of a subject antibody (e.g., an anti-C1s antibody); about 0.001% to about 1% of at least one. surfactant; about 1 mM to about 100 mM of a buffer; optionally about 10 mM to about 500 mM of a stabilizer; and about 5 mM to about 305 mM of a tonicity agent; and has a pH of about 4.0 to about 7.0.

As another example, a suitable parenteral formulation is a liquid or lyophilized formulation comprising: about 1 mg/mL to about 200 mg/mL of an anti-C1s antibody; 0.04% Tween 20 w/v; 20 mM L-histidine; and 250 mM sucrose; and has a pH of 5.5.

As another example, a subject parenteral formulation comprises a lyophilized formulation comprising: 1) 15 mg/mL of an anti-C1s antibody; 0.04% Tween 20 w/v; 20 mM L-histidine; and 250 mM sucrose; and has a pH of 5.5; or 2) 75 rug/mL of a subject antibody; 0.04% Tween 20 w/v; 20 mM L-histidine; and 250 mM sucrose; and has a pH of 5.5;or 3) 75 mg/mL of an anti-C1s antibody; 0.02% Tween 20 w/v; 20 mM L-histidine; and 250 mM sucrose; and has a pH of 5.5; or 4) 75 mg/mL of an anti-C1s antibody; 0.04% Tween 20 w/v; 20 mM L-histidine; and 250 mM trehalose; and has a pH of 5.5; or 5) 75 mg/mL of an anti-C1s antibody; 0.02% Tween 20 w/v; 20 mM L-histidine; and 250 mM trehalose; and has a pH of 5.5.

As another example, a suitable parenteral formulation is a liquid formulation comprising: 1) 7.5 mg/mL of an anti-C1s antibody; 0.02% Tween 20 w/v; 120 mM L-histidine; and 250 125 mM sucrose; and has a pH of 5.5; or 2) 37.5 mg/mL of an anti-C1s antibody; 0.02% Tween 20 w/v; 10 mM L-histidine; and 125 mM sucrose; and has a pH of 5.5; or 3) 37.5 mg/mL of an anti-C1s antibody; 0.01% Tween 20 w/v; 10 mM L-histidine; and 125 mM sucrose; and has a pH of 5.5; or 4) 37.5 mg/mL of an and-C1s antibody; 0.02% Tween 20 w/v; 10 mM L-histidine; 125 mM trehalose; and has a pH of 5.5; or 5) 37.5 mg/mL of an anti-C1s antibody; 0.01% Tween 20 w/v; 10 mM L-histidine; and 125 mM trehalose;

and has a pH of 5.5; or 6) 5 mg/mL of an anti-C1s antibody; 0.02% Tween 20 w/v; 2.0 mM L-histidine; and 250 mM trehalose; and has a pH of 5.5; or 7) 75 mg/mL of an anti-C1s antibody; 0.02% Tween 20 w/v; 20 mM L-histidine; and 250 mM mannitol; and has a pH of 5.5; or 8) 75 mg/mL of an anti-C1s antibody; 0.02% Tween 20 w/v; 20 mM L histidine; and 15 140 mM sodium chloride; and has a pH of 5.5;or 9) 150 mg/mL of an anti-C1s antibody;

0.02% Tween 20 w/v; 20 mM L-histidine; and 250 mM trehalose; and has a pH of 5.5; or 10) 150 mg/mL of an anti-C1s antibody; 0.02% Tween 20 w/v; 20 mM L-histidine; and 250 mM mannitol; and has a pH of 5.5; or 11) 150 mg/mL of an anti-C1s antibody; 0.02% Tween 20 w/v; 20 mM L-histidine; and 140 mM sodium chloride; and has a pH of 5.5; or 12) 10 20 mg/mL of an and-C1s antibody; 0.01% Tween 20 w/v; 20 mM L-histidine; and 40 mM sodium chloride; and has a pH of 5.5.

Suitable excipient vehicles are, for example, water, saline, dextrose, glycerol, ethanol, or the like, and combinations thereof. in addition, if desired, the vehicle can contain minor amounts of auxiliary substances such as wetting or emulsifying agents or pH buffering agents. Actual methods of preparing such dosage forms are known, or will be apparent, to those skilled in the art. See, e.g., Remington's Pharmaceutical Sciences, Mack Publishing Company, Easton, Pennsylvania, 17th edition, 1985. The composition or formulation to be administered will, in any event, contain a quantity of a subject antibody adequate to achieve the desired state in the subject being treated.

The pharmaceutically acceptable excipients, such as vehicles, adjuvants, carriers or diluents, are readily available to the public. Moreover, pharmaceutically acceptable auxiliary , substances, such as pH adjusting and buffering agents, tonicity adjusting agents, stabilizers, wetting agents and the like, are readily available to the public.

### Dosages

A proximal classical complement pathway inhibitor is administered in a therapeutically effective amount. The inhibitor can be administered to the subject with a certain frequency and for a period of time so as to achieve the desired therapeutic effect (e.g., reduction or prevention of homolysis). The frequency of administration may also be adjusted according to various parameters, including but not limited to, the clinical response, the plasma half-life of the inhibitor, and the levels of the inhibitor in a body fluid, such as, blood, plasma, serum, or synovial fluid. To guide adjustment of the frequency of administration, levels of the inhibitor in the body fluid may be monitored during the course of treatment.

Anti-C1s antibodies

In one aspect, the method comprising administering an anti-C1s antibody (e.g., sutimlimab) to the subject, where the anti-C1s antibody is administered in an effective amount of at least 4 g, at least 4.5 g, at least 5 g, at least 5.5 g, at least 6 g, at least 6.5 g, at least 7 g, at least 7.5 g, at least 8 g, at least 8.5 g, at least 9 g, at least 9.5 g, or at least 10 g.

In some embodiments, the anti-C1s antibody (e.g., sutimlimab) is administered in an effective amount between about 5.5 g and about 10 g, about 5.5 g and about 9.5 g, about 5.5 g and about 9 g, about 5.5 g and about 8.5 g, about 5.5 g and about 8 g, about 5.5 g and about 7.5 g, about 5.5 g and about 7 g, about 5.5 g and about 6.5 g, or about 5.5 g and about 6 g. In some embodiments, the anti-C1s antibody is administered in an amount between about 4.5 g and about 8.5 g, about 4.5 g and about 8 g, about 4.5 g and about 7.5 g, about 4.5 g and about 7 g, about 4.5 g and about 6.5 g, about 4.5 g and about 6 g, about 4.5 g and about 5,5 g, or about 4.5 g and about 5 g. In some embodiments, the anti-C1s antibody is administered in an amount between about 7.5 g and about 12 g, about 7.5 g and about 11.5 g, about 7.5 g and about 11 g, about 7.5 g and about 10.5 g, about 7.5 g and about 10 g, about 7.5 g and about 9.5 g, about 7.5 g and about 9 g, about 7.5 g and about 8.5 g, or about 7.5 g and about 8 g.

In some embodiments, the subject for the present method weighs 75kg or more and the anti-C1s antibody (e.g., sutimlimab) is administered in an effective amount of about 7.5 g. In other aspects, the subject for the present method weighs less than 75kg and the anti-C1s antibody (e.g., sutimlimab) is administered in an effective amount of about 6.5 g.

In some embodiments, the anti-C1s antibody (e.g., sutimlimab) is administered in an effective amount, between about 6.5 g and about 7.5 g.

In some aspects, the serum concentration of the anti-C1s antibody (e.g., sutimlimab) after the administration is at least 20 μg/mL, at least 25 μg/mL, at least 30 μg/mL, at least 35 μg/mL, at least 40 μg/mL, at least 45μg/mL, at least 50 μg/mL, at least 55 μg/mL, at least 60 μg/mL, at least 65 μg/mL, at least 70 μg/mL, at least 75 μg/mL, at least 80 μg/mL, at least 85 μg/mL, at least 90 μg/mL, at least 95 μg/mL, at least 100 μg/mL, at least 120 μg/mL, at least 130 μg/mL, at least 140 at least 150 μg/mL, at least 160 μg/mL, at least 170 μg/mL, at least 180 μg/mL, at least 190 μg/mL, at least at least 191 μg/mL, at least 192 μg/mL, at least 193 μg/mL, at least 194 μg/mL, at least 195 μg/mL, at least 196 μg/mL, at least 197 μg/mL, at least 198 μg/mL, at least 199 μg/mL, at least 200 μg/mL, at least 192 μg/mL, at least 384 μg/mL, at least 576 μg/mL, at least 768 μg/mL, or at least 800 μg/mL. In some embodiments of the disclosure, the serum concentration of the anti-C1s antibody after the administration is between about 800 μg/mL and about 200 μg/mL, between about 768 μg/mL and about 192 μg/mL, between about 768 μg/mL and about 384 μg/mL, between about 768 μg/mL and about 576 μg/mL, between about 576 μg/mL and about 192 μg/mL, between about 576 μg/mL and about 384 μg/mL, between about 384 μg/mL and about 192 μg/mL, between about 20 μg/mL, and about 100 μg/mL, about 20 μg/mL and about 90μg/mL, about 20 μg/mL and about 80 μg/mL, about 20 μg/mL and about 70 about 20 μg/mL and about 70 μg/mL, about 20 μg/mL and about 60 μg/mL, about 20 μg/mL and about 50 μg/mL, about 20 μg/mL and about 40 μg/mL, or about 20 μg/mL and about 30 μg/mL. In some embodiments, the serum concentration of the anti-C1s antibody after the administration is at least 20 μg/mL. In some embodiments, the serum concentration of the anti-C1s antibody after the administration is at least 100 μg/mL. In some embodiments, the serum concentration of the anti-C1s antibody after administration is at least 192 μg/mL. In some embodiments, the serum concentration of the anti-C1s antibody after administration is at least 384 μg/mL. In some embodiments, the serum concentration of the anti-C1s antibody after administration is at least 576 μg/mL. In some embodiments, the serum concentration of the anti-C1s antibody after administration is at. least 768 μg/mL.

In some aspects, maintaining a therapeutic serum concentration of the anti-C1s antibody (e.g., sutimlimab) comprises maintaining a serum concentration of the anti-C1s antibody of at least 20μg/mL, at least 25 μg/mL, at least 30 μg/mL, at least 35 μg/mL, at least 40 μg/mL, at least 45 μg/mL, at least 50 μg/mL, at least 55 μg/mL, at least 60 μg/mL, at least 65 μg/mL, at least 70 μg/mL, at least 75 μg/mL, at least 80 μg/mL, at least 85 μg/mL, at least 90 μg/mL, at least 95 μg/mL, at least 100 μg/mL, at least 120 μg/mL, at least 130 μg/ml,, at least 140 μg/mL, at least 150 at least 160 μg/mL, at least 170 μg/mL, at least 180 μg/mL, at least 190 μg/mL, at least 191 μg/mL, at least 192 μg/mL, at least 193 μg/mL, at least 194 μg/mL, at least 195 μg/mL, at least 196 μg/mL, at least 197 μg/mL, at least 198 μg/mL, at least 199 μg/mL, at least 200 μg/mL, at least 384 μg/mL, at least 576 μg/mL, at least 768 μg/mL, or at least 800 μg/mL. In some embodiments of the disclosure, maintaining the therapeutic serum concentration of the anti-C1s antibody comprises maintaining a serum concentration of between about 800 μg/mL and about 200 μg/mL, between about 768 μg/mL and about 192 μg/mL, between about 768 μμg/mL and about 384 μg/mL, between about 768 μg/mL and about 576 μg/mL, between about 576 μg/mL and about 192 μg/mL, between about 576 μg/mL and about 384 μg/mL, between about 384 μg/mL and about 192 μg/mL, between about 20 μg/mL and about 100 μg/mL, about 20 μg/mL and about 90 μg/mL, about 20 μg/mL and about 80 μg/mL, about 20 μg/mL and about 70 μg/mL, about 20 μg/mL and about 70 μg/mL, about 20 μg/mL, and about 60 μg/mL, about 20 μg/mL and about 50 μg/mL, about 20 μg/mL and about 40 μg/mL, or about 20 μg/mL and about 30 μg/mL. In some embodiments, maintaining the therapeutic serum concentration of the anti-C1s antibody comprises maintaining a serum concentration of at least 20 μg/mL. In some embodiments, maintaining the therapeutic serum concentration of the anti-C1s antibody comprises maintaining a serum concentration of at least 100 μg/mL. In some embodiments, maintaining the therapeutic serum concentration of the anti-C1s antibody comprises maintaining a serum concentration of at least 192 μg/mL. In some embodiments, maintaining the therapeutic serum concentration of the anti-C1s antibody comprises maintaining a serum concentration of at least 384 μg/mL. In some embodiments, maintaining the therapeutic serum concentration of the anti-C1s antibody comprises maintaining a serum concentration of at least 576 μg/mL. In some embodiments, maintaining the therapeutic serum concentration of the anti-C1s antibody comprises maintaining a serum concentration of at least 768 μg/mL.

In some aspects, the major surgery is performed when the subject's serum concentration of the anti-C1s antibody (e.g., sutimlimab) is (or predicted to be) effective to reduce or prevent hemolysis. In some embodiments, the major surgery is performed on a day when the subject's serum concentration of the anti-C1s antibody is (or predicted to be) at least 20 μg/mL, at least 25 μg/mL, at least 30 μg/mL, at least 35 μg/mL, at least 40 μg/mL, at least 45 μg/mL, at least 50 μg/mL, at least 55 μg/mL, at least 60 μg/mL, at least 65 μg/mL, at least 70 μg/mL, at least 75 μg/mL, at least 80 μg/mL, at least 85 μg/mL, at least 90 μg/nit, at least 95 μg/mL, at least 100 μg/mL, at least 120 μg/mL, at least 130 μg/mL, at least 140 μg/mL, at least 150 μg/mL, at least 160 μg/mL, at least 170 μg/mL, at least 180 μg/mL, at least 190 μg/mL, at least 191 μg/mL, at least 192 μg/mL, at least 193 μg/mL, at least 194 μg/mL, at least 195 μg/mL, at least 196 μg/mL, at least 197 μg/mL, at least 198 μg/mL, at least 199 μg/mL, at least 200 μg/mL, at least 384 μg/mL, at least 576 μg/mL, at least 768 μg/mL, or at least 800 μg/mL. In some embodiments, the major surgery is performed on a day when the subject's serum concentration of the anti-C1s antibody is (or predicted to be) between about 800 lag/mL and about 200 μg/mL, between about 768 μg/ml, and about 192 μg/mL, between about 768 μg/mL and about 384 μg/mL, between about 768 μg/mL and about 576 μg/mL, between about 576 μg/ml, and about 192 μg/mL, between about 576 vg/mL and about 384 μg/mL, between about 384 μg/mL, and about 192 μg/mL, between about 20 μg/mL and about 100 μg/mL, about 20 μg/mL and about 90 μg/mL, about 20 μg/mL and about 80 μg/mL, about 20 μg/mL and about 70 μg/mL, about 20 μg/mL and about 70 μg/mL, about 20 μg/mL, and about 60μg/mL, about 20 μg/mL and about 50 μg/mL, about 20 lag/mL and about 40 μg/mL, or about 20 and about 30 μg/mL. In some embodiments, the major surgery is performed on a day when the subject's serum concentration of the anti-C1s antibody is (or predicted to be) at least 20 μg/mL. In some embodiments, the major surgery is performed on a day when the subject's serum concentration of the anti-C1s antibody is (or predicted to be) at least 100 μg/mL. In some embodiments, the major surgery is performed on a day when the subject's serum concentration of the anti-C1s antibody is (or predicted to be) at least 192 μg/mL. In some embodiments, the major surgery is performed on a day when the subject's serum concentration of the anti-C1s antibody is (or predicted to be) at least 384 μg/mL. In some embodiments, the major surgery is performed on a day when the subject's serum concentration of the anti-C1s antibody is (or predicted to be) at least 576 μg/mL. In some embodiments, the major surgery is performed on a day when the subject's serum concentration of the anti-C1s antibody is (or predicted to be) least 768 μg/mL.

The serum concentration of the anti-C1s antibody (e.g., sutimlimab) in the subject can be measured using techniques known in the art. In some embodiments, the and-C1s antibody is measured using a direct binding Enzyme-Linked Immunosorbent Assay (ELISA). In some embodiments, the anti-C1s antibody is measured using an indirect ELISA. In some embodiments, the and-C1s antibody is measured using a sandwich ELISA. In some embodiments the anti-C1s antibody is measured using a competitive ELISA.

In some aspects, an effective dose of an anti-C1s antibody (e.g., sutimlimab) is at least 45 mg/kg, at least 50 mg/kg, at least 55 mg/kg, at least 60 mg/kg, at least 65 mg/kg, at least 70 mg/kg, at least 75 mg/kg, at least 80 mg/kg, at least 85 mg/kg, at least 90 mg/kg, at least 95 mg/kg, or at least 100 mg/kg. In some embodiments, the effective dose of the anti-C1s antibody is at least 60 mg/kg.

In some embodiments, the effective dose of the anti-C1s antibody (e.g., sutimlimab) is between about 60 mg/kg and about 100 mg/kg, about 60 mg/kg and about 95 mg/kg, about 60 mg/kg and about 90 mg/kg, about 60 mg/kg and about 85 mg/kg, about 60 mg/kg and about 80 mg/kg, about 60 mg/kg and about 75 mg/kg, about 60 mg/kg and about 70 mg/kg, or about 60 mg/kg and about 65 mg/kg. In some embodiments, the effective dose of the anti-C1s antibody is between about 45 mg/kg and about 85 rag/kg, about 45 mg/kg and about 80 mg/kg, about 45 mg/kg and about 75 mg/kg, about 45 mg/kg and about 70 mg/kg, about 45 mg/kg and about 65 mg/kg, about 45 mg/kg and about 60 mg/kg, or about 45 mg/kg and about 50 mg/kg. In some embodiments, the effective dose of the anti-C1s antibody is between about 85 mg/kg and about 150 mg/kg, about 85 mg/kg and about 145 mg/kg, about 85 mg/kg and about 140 mg/kg, about 85 mg/kg and about 135 mg/kg, about 85 mg/kg and about 130 mg/kg, about 85 mg/kg and about 125 mg/kg, about 85 mg/kg and about 125 mg/kg, about 85 mg/kg and about 120 mg/kg, about 85 mg/kg and about 115 mg/kg, about 85 mg/kg and about 110 mg/kg, about 85 mg/kg and about 105 mg/kg, about 85 mg/kg and about 100 mg/kg, about 85 mg/kg and about 95 mg/kg, or about 85 mg/kg and about 90 mg/kg.

In some embodiments, the effective dose for the present methods is about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, about 105 mg/kg, about 110 mg/kg, about 115 mg/kg, about 120 mg/kg, about 125 mg/kg, about 130 mg/kg, about 135 mg/kg, about 140 mg/kg, about 145 mg/kg, or about 150 mg/kg.

### Route of Administration

A proximal classical complement pathway inhibitor is administered to a subject using any available method and route suitable for drug delivery, including in vivo and ex vivo methods, as well as systemic and localized routes of administration.

Conventional and pharmaceutically acceptable routes of administration include intranasal, intramuscular, intratracheal, intrathecal, intracranial, subcutaneous, intradermal, topical, intravenous, intraperitoneal, intraarterial (e.g., via the carotid artery), spinal or brain delivery, rectal, nasal, oral, and other enteral and parenteral routes of administration. Routes of administration can be combined, if desired, or adjusted depending upon the antibody and/or the desired effect. An inhibitor (e.g., an anti-C1s antibody) composition can be administered in a single dose or in multiple doses. In some embodiments, an inhibitor is administered orally. In some embodiments, inhibitor is administered subcutaneously. In some embodiments, an inhibitor (e.g., an anti-C1s antibody) is administered intramuscularly. In some embodiments, an anti-C1s antibody is administered intravenously.

An inhibitor (e.g., an anti-C1s antibody) can be administered to a host using any available conventional methods and routes suitable for delivery of conventional drugs, including systemic or localized routes. In general, routes of administration contemplated by the disclosure include, but are not necessarily limited to, enteral, parenteral, or inhalational routes.

Parenteral routes of administration other than inhalation administration include, but are not necessarily limited to, topical, transdermal, subcutaneous, intramuscular, intraorbital, intracapsular, intraspinal, infrasternal, intrathecal, and intravenous routes, i.e., any route of administration other than through the alimentary canal. Parenteral administration can be carried to effect systemic or local delivery of a subject antibody. Where systemic delivery is desired, administration typically involves invasive or systemically absorbed topical or mucosal administration of pharmaceutical preparations. In some embodiments, the dose is administered as intravenous infusion over 1 hour. Intravenous infusion can take place within clinic or home setting.

In some embodiments, inhibitor (e.g., an anti-C1s antibody) is administered by injection and/or delivery, e.g., to a site in a brain artery or directly into brain tissue. An inhibitor (e.g., an anti-C1s antibody) can also be administered directly to a target site e.g., by biolistic delivery to the target site.

A variety of subjects (wherein the term “subject” is used interchangeably herein with the terms “individual,” and “patient”) are treatable according to the subject methods. Generally, such subjects are “mammals” or “mammalian,” where these terms are used broadly to describe organisms which are within the class mammalia, including the orders carnivore (e.g., cats), herbivores (e.g., cattle, horses, and sheep), omnivores (e.g., dogs, goats, and pigs), rodentia (e.g., mice, guinea pigs, and rats), and primates (e.g., humans, chimpanzees, and monkeys). In some embodiments, the subject is an individual that has a complement system, such as a mammal, fish, or invertebrate. In some embodiments, the. subject is a complement system-containing mammal, fish, or invertebrate companion animal, agricultural animal, work animal, zoo animal, or lab animal. In some embodiments, the subject is human.

In some embodiments, the present methods have no limitation of use associated with anemia severity, transfusion history, or prior treatment experience.

### EXAMPLES

This example describes a case study to assess the efficacy of sutimlimab in a patient diagnosed with CAD undergoing major surgery. A Caucasian male in his late sixties diagnosed with CAD had a known history of coronary heart disease (CHD), presenting 10 years earlier with myocardial infarction treated with three stents in the right coronary artery (RCA). Reoccurrence of symptoms led to a new angiography and placement of a stent in the left anterior descending artery (LAD) one year later and two more additional LAD stents eight years after. At CAD diagnosis 1 year after his last cardiac surgery, he was severely anemic and required regular RBC transfusions, He was enrolled in the Phase 3 Cardinal trial, an open-label, single-arm study of sutimlimab, a selective inhibitor of complement C1s in patients with CAD with recent histories of transfusions. His hemoglobin levels increased from below 10 with regular RBC transfusions to above 12.0 g/dl without transfusion requirements, but 9 months later he developed progressive dyspnea which led to an angiography that demonstrated three-vessel disease with proximal and distal stenosis in the LAD and the circumflex artery (CX), indicating the need for open cardiac surgery.

Currently, treatment for CAD involves one of two options: plasma exchange to remove IgM or chemoimmunotherapy agents to reduce antibody production. Although plasmapheresis is highly effective in removing CA, the effect is short-lived since IgM production continues. Chemoimmunotherapy may lead to long-term remission, but time to response is delayed up to several months, and severe neutropenia and infectious complications are seen in at least 20% of patients. As the patient required immediate treatment and t prior to inclusion in the study had experienced a drop in hemoglobin level during acute phase episodes, the risk of exacerbating hemolysis during the postoperative phase was considered to be significant. Thus, none of the available options were considered to be suitable for the patient.

Sutimlimab is a selective inhibitor of complement C1s that selectively blocks classical complement activation but leaves the alternative and lectin pathways of the complement cascade intact. Sutimlimab is highly efficient in blocking complement-mediated hemolysis, and data from phase I and Phase 3 trials demonstrate rapid and complete blockade of hemolysis in CAD. However, non-complement mediated symptoms of RBC agglutination may not be relieved. Cold-induced, IgM-mediated agglutination can be prevented by keeping the body temperature and circulating fluids warm, above CA thermal amplitude at 37° C. throughout the surgery. The inventors found that continuing sutimlimab might sufficiently block exacerbation of hemolysis via the classical complement pathway during surgery as well as the postoperative period.

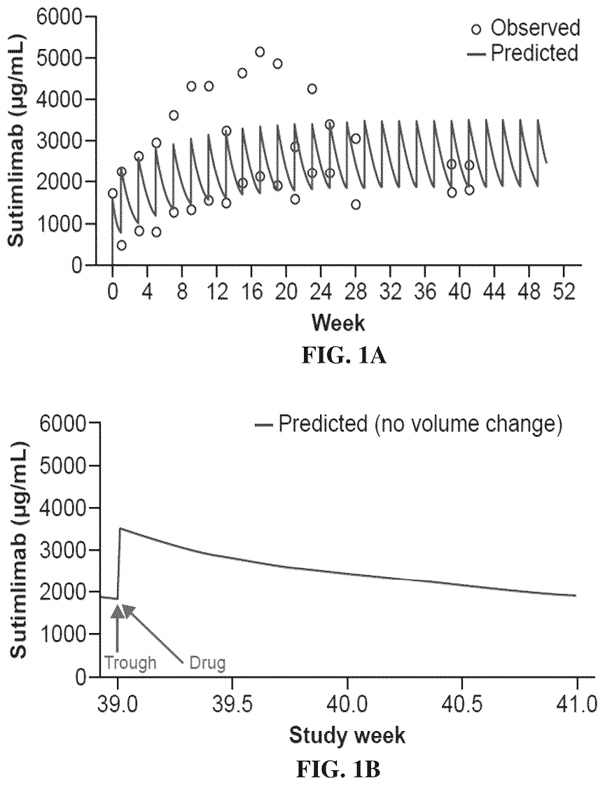

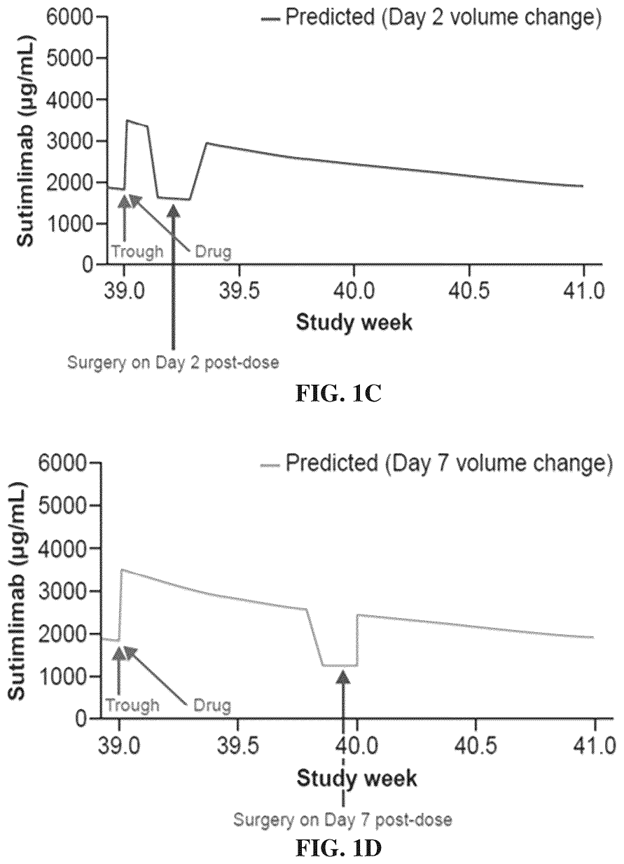

Significant dilution of circulating sutimlimab was expected during open cardiac surgery as the patient would have to be connected to extracorporeal circulation, e.g., the heart-lung machine, which would be primed with 1300 mL of solution (Mannitol, electrolyte and buffer solution). A blood loss of approximately 500 mL and infusion of 100-2000 mL of fluid was further anticipated. Using these data together with the patient's previous PK/PD profile (FIG. 1), it was expected that the intraoperative concentration of sutimlimab (FIG. 1B) would be 4 times higher than what is needed to sufficiently suppress hemolysis if the surgery was done 2 days after a routine sutimlimab administration.

The patient was admitted for cardiac surgery on day 0 and given a routine infusion of sutimlimab, given as a 6.5 g dose as appropriate for the patient's weight. He then underwent coronary artery bypass grafting (CABG) on day 2. Precautions were taken to keep the operating room temperature high to avoid cooling of the patient. The fluids in the heart-lung machine were kept at 37° C. CABG was performed through median sternotomy with extracorporeal circulation through standard cannulation (at 37° C.). Myocardial protection was delivered with antegrade warm (37° C.) blood cardioplegia. The patient received two coronary grafts; the left anterior mammary artery (LIMA) to LAD and a saphenous vein graft to CX. To avoid replacement of CIA, no plasma products or coagulations factors were given during surgery. Blood samples were collected at admission, prior to surgery, immediately prior to and after extracorporeal circulation, and 24 hours after surgery.

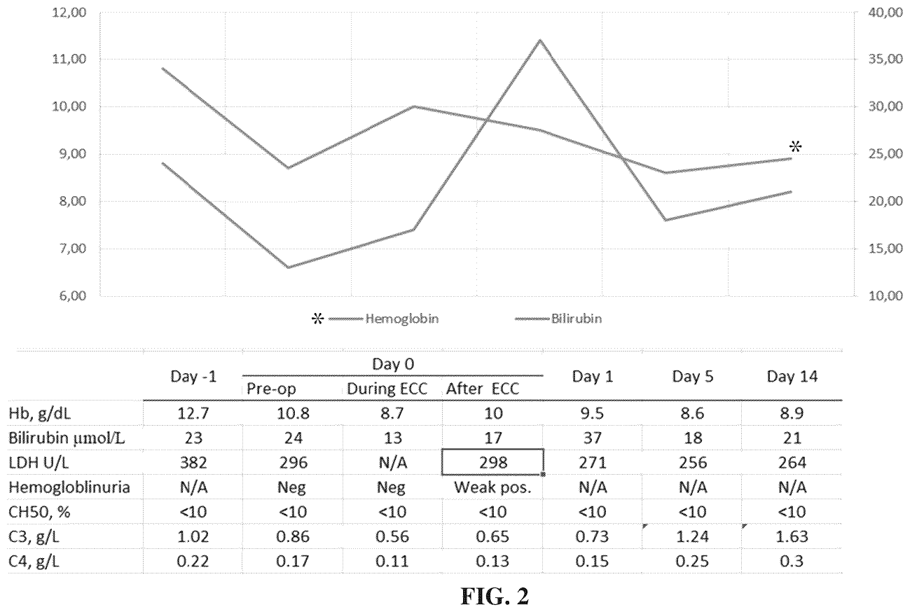

As expected, a slight decrease in hemoglobin levels were observed when the Fluids in the heart-lung machine diluted the patient's blood. Hemoglobin levels remained stable throughout the surgery with no signs of hemolysis, e.g., stable lactate dehydrogenase levels and no hematuria or discoloration of plasma (FIG. 2). The early postoperative course was uneventful, and the patient was discharged to a local hospital after five days, well mobilized and in a good condition. As expected after major surgery, a significant inflammatory response was observed, with increased C-reactive protein (CRP) levels that peaked 2 days after surgery (CRP 225 mg/L), but with no apparent signs of infection. Despite this inflammatory response, no breakthrough hemolysis was observed and levels for LDH, bilirubin, and hemoglobin remained stable. At routine follow up on day 14, he was fully mobilized, with a stable hemoglobin level and no signs of hemolysis (FIG. 2). Total complement activity (CH50), monitored to assess the suppression of the classical complement pathway by sutimlimab, was completely suppressed prior to surgery after sutimlimab infusion. CH50 remained suppressed during surgery and the early post-operative period and was still suppressed at the follow-up visit 2 weeks later, indicating a continuous and complete inhibition of the classical complement pathway.

The current case demonstrates that even in situations with a significant acute phase reaction and simultaneous shift in volume status, sutimlimab can efficiently block hemolysis for a longer period. In addition to preventing hemolytic exacerbation, the prophylactic C1s inhibition also seemed to efficiently prevent activation of the complement system despite a marked acute phase reaction following major surgery. The patient had achieved a higher sutimlimab level and suppression of the classical complement cascade after more than 52 weeks with sutimlimab infusions, compared to only a single infusion (FIG. 1A). Sutimlimab naive patients could possibly require a higher dose prior to major surgery, than used in the current patient. As sutimlimab leaves the alternative and terminal pathways unaffected, an increase in infectious complications is unlikely. Hence, sutimlimab is potentially a new and safe tool for patients with CAD undergoing major surgery.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean ±10% of the recited numerical value.

Where a range of values is provided, each value between and including the upper and lower ends of the range are specifically contemplated and described herein.

1. A method of reducing or preventing hemolysis in a subject in need thereof undergoing a major surgery, comprising maintaining in the subject a therapeutic serum concentration of a proximal classical complement pathway inhibitor, wherein the subject has cold agglutinin disease (CAD) and the therapeutic serum concentration is effective to reduce or prevent hemolysis.

2. The method of claim 1, wherein the maintaining comprises administering to the subject a maintenance dose of the proximal classical complement pathway inhibitor before the subject undergoes the major surgery, wherein the dose is effective to maintain the therapeutic serum concentration during the major surgery.

3. The method of claim 2, wherein the maintaining comprises administering to the subject the maintenance dose of the proximal classical complement pathway inhibitor within seven days, within three days, within two days, or within one day of the subject undergoing the major surgery of the subject undergoing the major surgery.

4. (canceled)

5. The method of claim 1, further comprising assessing in the subject a therapeutic serum concentration of the proximal classical complement pathway inhibitor.

6. The method of claim 1, wherein the maintaining comprises administering to the subject at least one additional dose of the proximal classical complement pathway inhibitor before, during and/or after the subject undergoes the major surgery.

7. A method of reducing or preventing hemolysis in a subject in need thereof undergoing a major surgery, comprising performing the major surgery on the subject when the subject has a proximal classical complement pathway inhibitor serum concentration effective to reduce or prevent hemolysis, wherein the subject has cold agglutinin disease (CAD) and has been undergoing treatment with the proximal classical complement pathway inhibitor.

8. The method of claim 7, wherein the treatment comprises administration of at least one loading dose and at least one maintenance dose of the proximal classical complement pathway inhibitor.

9. The method of claim 7, wherein the method comprises (a) administering to the subject at least one maintenance dose of the proximal classical complement pathway inhibitor, and (b) performing the major surgery on the subject within seven days of administering the at least one maintenance dose of the proximal classical complement pathway inhibitor.

10. The method of claim 9, further comprising, prior to (a), administering to the subject at least one loading dose of a proximal classical complement pathway inhibitor.

11. The method of claim 9 [[or 10]], wherein (b) comprises performing the major surgery on the subject within three days, within two days, or within one day of administering the at least one maintenance dose of the proximal classical complement pathway inhibitor.

12. A method of reducing or preventing hemolysis in a subject in need thereof undergoing a major surgery, comprising:
assessing a serum concentration of a proximal classical complement pathway inhibitor in a subject who has cold agglutinin disease (CAD) and is undergoing treatment with a proximal classical complement pathway inhibitor; and
performing the major surgery on the subject within seven days of the assessing.

13. The method of claim 12, comprising performing the major surgery on the subject within three days, within two days, or within one day of the assessing.

14. The method of claim 12, comprising assessing the serum concentration of the proximal classical complement pathway inhibitor before, during, and/or after the major surgery.

15. The method of claim 12, further comprising administering to the subject at least one dose of the proximal classical complement pathway inhibitor before, during and/or after performing the major surgery on the subject.

16. The method of claim 1, wherein the major surgery is a major cardiac surgery, optionally wherein the major cardiac surgery is coronary artery bypass graft (CABG) surgery.

17. (canceled)

18. The method of claim 1, wherein (i) the major surgery is associated with a drop in body temperature and/or hypoxia, and/or (ii) the major surgery involves hemodilution and/or extracorporeal circulation.

19. (canceled)

20. The method of claim 1, wherein the proximal classical complement pathway inhibitor is a C1s inhibitor, a C1r inhibitor, a C1q inhibitor, a C2 inhibitor, or a C4 inhibitor, optionally wherein the proximal classical complement pathway inhibitor is a C1s inhibitor.

21. (canceled)

22. The method of claim 1, wherein the inhibitor is an antibody, optionally wherein the antibody is sutimlimab.

23. (canceled)

24. The method of claim 22, wherein sutimlimab is administered in an amount of about 5 grams to about 8 grams, optionally wherein sutimlimab is administered in an amount of about 6.5 grams to about 7.5 grams, and further optionally wherein the subject weighs less than 75 kilograms, and the amount is 6.5 grams, or the subject weighs 75 kilograms or more, and the amount is 7.5 grams.

25-26. (canceled)

27. The method of claim 1, wherein the therapeutic serum concentration of the inhibitor is at least 90 μg/mL, at least 100 μg/mL, or at least 192 μg/mL.

28-29. (canceled)


SEQUENCE LISTING (24 sequences)

SEQ ID NO: 1 - protein, 445 aa, synthetic construct
  REGION 1..445: note = Synthetic
EVQLVESGGGLVKPGGSLRLSCAASGFTFSNYAMSWVRQAPGKGLEWVATISSGGSHTYYLDSVKGRFTISRDNSKNTLYLQMNSLRAEDTALYYCARLFTGYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 216 aa, synthetic construct
  REGION 1..216: note = Synthetic
QIVLTQSPATLSLSPGERATMSCTASSSVSSSYLHWYQQKPGKAPKLWIYSTSNLASGVPSRFSGSGSGTDYTLTISSLQPEDFATYYCHQYYRLPPITFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 118 aa, synthetic construct
  REGION 1..118: note = Synthetic
EVQLVESGGGLVKPGGSLRLSCAASGFTFSNYAMSWVRQAPGKGLEWVATISSGGSHTYYLDSVKGRFTISRDNSKNTLYLQMNSLRAEDTALYYCARLFTGYAMDYWGQGTLVTVSS

SEQ ID NO: 4 - protein, 109 aa, synthetic construct
  REGION 1..109: note = Synthetic
QIVLTQSPATLSLSPGERATMSCTASSSVSSSYLHWYQQKPGKAPKLWIYSTSNLASGVPSRFSGSGSGTDYTLTISSLQPEDFATYYCHQYYRLPPITFGQGTKLEIK

SEQ ID NO: 5 - protein, 5 aa, synthetic construct
  REGION 1..5: note = Synthetic
NYAMS

SEQ ID NO: 6 - protein, 17 aa, synthetic construct
  REGION 1..17: note = Synthetic
TISSGGSHTYYLDSVKG

SEQ ID NO: 7 - protein, 9 aa, synthetic construct
  REGION 1..9: note = Synthetic
LFTGYAMDY

SEQ ID NO: 8 - protein, 12 aa, synthetic construct
  REGION 1..12: note = Synthetic
TASSSVSSSYLH

SEQ ID NO: 9 - protein, 7 aa, synthetic construct
  REGION 1..7: note = Synthetic
STSNLAS

SEQ ID NO: 10 - protein, 10 aa, synthetic construct
  REGION 1..10: note = Synthetic
HQYYRLPPIT

SEQ ID NO: 11 - protein, 446 aa, synthetic construct
  REGION 1..446: note = Synthetic
QVQLVQSGAEVKKPGASVKLSCTASGFNIKDDYIHWVKQAPGQGLEWIGRIDPADGHTKYAPKFQVKVTITADTSTSTAYLELSSLRSEDTAVYYCARYGYGREVFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 12 - protein, 218 aa, synthetic construct
  REGION 1..218: note = Synthetic
DIVLTQSPDSLAVSLGERATISCKASQSVDYDGDSYMNWYQQKPGQPPKILIYDASNLESGIPARFSGSGSGTDFTLTISSLEPEDFAIYYCQQSNEDPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 119 aa, synthetic construct
  REGION 1..119: note = Synthetic
QVQLVQSGAEVKKPGASVKLSCTASGFNIKDDYIHWVKQAPGQGLEWIGRIDPADGHTKYAPKFQVKVTITADTSTSTAYLELSSLRSEDTAVYYCARYGYGREVFDYWGQGTTVTVSS

SEQ ID NO: 14 - protein, 446 aa, synthetic construct; identical to SEQ ID NO: 11
  REGION 1..446: note = Synthetic

SEQ ID NO: 15 - protein, 5 aa, synthetic construct
  REGION 1..5: note = Synthetic
DDYIH

SEQ ID NO: 16 - protein, 17 aa, synthetic construct
  REGION 1..17: note = Synthetic
RIDPADGHTKYAPKFQV

SEQ ID NO: 17 - protein, 10 aa, synthetic construct
  REGION 1..10: note = Synthetic
YGYGREVFDY

SEQ ID NO: 18 - protein, 15 aa, synthetic construct
  REGION 1..15: note = Synthetic
KASQSVDYDGDSYMN

SEQ ID NO: 19 - protein, 7 aa, synthetic construct
  REGION 1..7: note = Synthetic
DASNLES

SEQ ID NO: 20 - protein, 9 aa, synthetic construct
  REGION 1..9: note = Synthetic
QQSNEDPWT

SEQ ID NO: 21 - protein, 327 aa, synthetic construct
  REGION 1..327: note = Synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 327 aa, synthetic construct
  REGION 1..327: note = Synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 23 - protein, 327 aa, synthetic construct
  REGION 1..327: note = Synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 24 - protein, 107 aa, synthetic construct [sequence omitted]